(19) United States
(12) Patent Application Publication
(10) Pub. No.: US 2025/0248926 A1
(43) Pub. Date: Aug. 7, 2025

(54) AN INJECTABLE MICROPARTICLE SYSTEM AND METHOD OF PREPARING THEREOF

(71) Applicant: AMRITA VISHWA VIDYAPEETHAM, Kochi, Kerala (IN)

(72) Inventors: Manzoor KOYAKUTTY, Kochi (IN); Devika DAS, Kochi (IN); Dhanya NARAYANAN, Kochi (IN); Ranjith RAMACHANDRAN, Ranjith (IN); Shantikumar NAIR, Kochi (IN); Sreelakshmy JAYACHANDRAN, Kochi (IN)

(73) Assignee: AMRITA VISHWA VIDYAPEETHAM, Kochi, Kerala (IN)

(21) Appl. No.: 18/880,874

(22) PCT Filed: Jul. 4, 2023
(86) PCT No.: PCT/IN2023/050652

§ 371 Date: Jan. 3, 2025

(30) Foreign Application Priority Data
Jul. 5, 2022 (IN) 202241038701

(51) Int. Cl. A61K 9/00; A61K 9/50; A61K 31/175; A61K 31/337; A61K 31/4412; A61K 31/495; A61K 31/513; A61K 31/704; A61K 31/7068

(52) U.S. Cl. CPC A61K 9/0024; A61K 9/5031; A61K 31/175; A61K 31/337; A61K 31/4412; A61K 31/495; A61K 31/513; A61K 31/704; A61K 31/7068

(57) ABSTRACT

Prolonged delivery of chemodrugs locally inside the brain with sufficient tissue-penetration is a critical requirement for treating various brain-diseases including malignant tumors. The present disclosure relates to an injectable microparticle system for brain-drug delivery. The injectable microparticle system comprising drug-loaded polymeric microparticles, wherein the drug-loaded polymeric microparticles are coated with an outer polymer gel layer enabling in-situ formation and releasing drug-polymer nanocomplexes of size <100 nm. The injectable microparticle system facilitates both deep tissue penetration for >2 cm as well as ‘sustained release’ of the drug for 15-30 days. The invention also discloses a method of producing said injectable microparticle system and the use of said injectable microparticle system in the treatment of brain tumor and other cancers.

### FIELD OF THE INVENTION

The present disclosure relates to the field of nano pharmaceutical formulation. Particularly, the present disclosure relates to an injectable microparticle system for brain-drug delivery. The injectable microparticle system facilitate both ‘deep tissue penetration’ as well as ‘sustained release’ of the drug. The invention also discloses a method of producing said injectable microparticle system and the formulation and the use of said injectable microparticle system in the treatment of brain tumor and other cancers.

### BACKGROUND OF THE INVENTION

Delivery of drugs into the brain has been a challenging mechanism due to the presence of tight junctions in the blood-brain barrier. Clinically, it was observed that only a minute fraction of drugs delivered through oral, or intravenous (IV) routes reaches the brain tissue, creating significant challenge in treating diseases such as brain tumor, Alzheimer's, and Parkinson's disease. In another aspect, systemic delivery of chemo drugs leads to undesired toxicity in other parts of the body, thereby affecting healthy tissues. Further, low permeation properties of hydrophobic drugs in lipid-rich brain hinders deep-penetration even if we inject drugs directly into the brain tissue.

Systemic delivery of nanoparticles have not made a significant difference compared to their drug alone counterparts, mainly due to their inability to protect the drug from proteins present within the blood, leading to protein corona formation, uptake by macrophages and presenting them to spleen and liver for its elimination. Because of this, the nanoparticles are not able to deliver the complete drug payload at the target location in brain through systemic delivery.

Nanomedicines fall in the size range between 1 to 1000 nm and they are known to deliver drugs to the desired location. Even though extensive research is conducted on nanomedicines, all of them are not translated, mainly due to their failure during clinical trials, owing to systemic toxicity, undesirable accumulation within the liver, quick clearance by the macrophages, and so on.

Glioblastoma Multiforme (GBM) is a Grade 4 brain tumor with median survival of patients <15 months from the time of diagnosis. Clinical treatment of GBM includes maximum surgical resection followed by chemotherapy and radiation. The major chemotherapeutic regimen is the systemic oral delivery of a DNA methylating agent, Temozolomide (oral) in combination with radiation therapy for 30 days.

In another mode of chemotherapy, drug loaded polymeric implant viz. Gliadel®, was used for localized drug delivery in the brain. Gliadel® is the only approved local drug delivery system for the treatment of brain tumor. Gliadel® is formed by loading the drug (Carmustine or BCNU) in a polymer blend of poly bis-(p-carboxyphenoxy) propane and Sebacic acid and compressed to the shape of a circular disc/wafer. 6-8 wafers are placed within the tumor resected cavity after surgical resection of tumor mass [2]. Upon degradation by pH or enzymes, the loaded drug is released into the tumor microenvironment. Gliadel® treated patients showed minor improvement in median survival by ˜2-3 months.

Even after the above-mentioned treatments, GBM was found to recur, typically from a distance of 2-3 cm from the boundary of primary resected tumor. The recurrence was primarily due to incomplete killing of residual cancer cells within 2-3 cm, suggesting that the drug, BCNU (1,3-Bis(2-chloroethyl)-1-nitrosourea), released from Gliadel® implant was not able to penetrate brain tissue up to 3 cm in sufficient concentration to eradicate remaining cancer cells. In another aspect, metastatic tumors occur deep within the brain in different new locations, which cannot be surgically removed due to sensitivity of the location. Such tumors can be removed only by chemotherapy or radiation, but limited tissue penetration of drugs pose a major challenge in such scenario.

Tissue-penetration of the drug molecule can be improved by using a carrier material that can slide through the brain tissue micro-environment. However, typical pore size of native brain tissue is 25-200 nm but majority of the pores fall <50 nm size scale. This means, nanoparticles loaded or complexed with drug of size <100 nm only can slide through the native pores. Typical nanoparticles prepared for drug delivery, using polymers such as Poly lactic-go-glycolic acid (PLGA), Poly lactic acid (PLA), Poly caprolactum (PCL) would be relatively large (200 to 500 nm) and not suitable for penetrating through the <50 nm size of brain pores. In addition, there should be less electrostatic interaction between the carrier nanomaterial or drug with the brain tissue such that the diffusion process is not hindered by strong electrostatic interaction.

Another challenge is associated with sustained release. Typically, very small NPs of size <100 nm will not be sufficiently large enough to hold the drug for longer duration as the drug will quickly diffuse out and/or such small NPs will be enzymatically degraded fast. However, larger nanoparticles or microparticles with higher bulk volume will be able to hold the drug for longer durations up to 15-30 days. This means, the requirement of deep-tissue penetration by <100 nm size scale and prolonged sustained release by way of larger microparticles contradicts each other.

A number of publications and patents reported method of drug delivery in brain. US010307372B2 describes the potential of providing a hydrophilic coating to Polystyrene (PS) nanoparticles with near neutral surface charge to enhance the penetration within the brain tissue. However, diffusion of drug was shown to be limited to micrometer-scale (200 μm in 60 minutes) using qualitative imaging in ex vivo mouse model, and no drug quantification was done in different sections of tissue. This difference in diffusion could possibly be attributed to variation in the preparation methods, wherein, the above patent used the methodology of chemically grafting polystyrene with PEG-co-polymer, which may take longer to degrade and diffuse.

Gliadel® (Eisai Inc. for Arbor Pharmaceuticals, LLC) (U.S. Pat. Nos. 4,888,176, 4,757,128) describes the preparation of carmustine loaded in polyanhydride wafer. A bulk release of BCNU from the wafer happens within the first 7 days, releasing almost the entire contents of drug. Whereas it was found that the released drug remains to be free molecule with very low tissue penetration of 1-5 mm. Patients also treated with Gliadel® showed tumor recurrence as against placebo treated controls, with just the exception of a delayed recurrence.

WO2017/192088 A1 describes the development of a stable preparation of Temozolomide (TMZ) based gel in a two step-process of dried powder consisting of a mixture of polysaccharide phosphate salts with Temozolomide followed by conversion into a drug loaded gel. However, this patent shows no data on the diffusion properties of the hydrogel in brain tissue.

Thus, it is imperative to develop novel drug delivery system which can facilitate drug release and penetration for >2 cm to treat micro-metastasis nodules. In addition, provide sustained release of drugs locally in the tumor affected area by creating continuous local concentration of drugs to kill residual cancer cells.

### SUMMARY OF THE INVENTION

The present invention provides an injectable microparticle system comprising drug-loaded polymeric microparticles, wherein the drug-loaded polymeric microparticles are coated with an outer polymer gel layer enabling in-situ formation and releasing drug-polymer nanocomplexes of size <100 nm. The injectable microparticle system facilitate both ‘deep tissue penetration’ as well as ‘sustained release’ of the drug.

The present invention also provides a method of producing said injectable microparticle system.

Further, the present invention provides a method of treating brain tumor by introducing injectable microparticle system into the brain tumor or tumor resected cavity or near to the tumor region in healthy brain tissue.

Furthermore, the present invention provides the use of injectable microparticle system in the treatment of brain tumor and other cancers.

Moreover, the present invention provides the use of an injectable microparticle system in combination with surgery, radiation therapy, photodynamic therapy, chemotherapy, immunotherapy, ultrasound therapy, radio-frequency ablation, tumor treating field therapy, cancer-vaccines, or combinations thereof.

### BRIEF DESCRIPTION OF THE DRAWINGS

The present invention is accompanied by following drawings, wherein:

FIG. 1A through FIG. 1C shows the schematic representation of (A) microparticles 1 releasing drug 3, which interacts with the outer polymer gel layer to form (B) drug-polymer nanocomplexes 2 of size <100 nm. FIG. 1C shows the injection of microparticle gel within the tumor cavity with microparticles staying at the surface releasing the drug in a sustained manner, whereas the nanocomplexes diffuse >2 cm.

FIG. 2 represents the drawing depicting the preparation of free drug loaded in microparticles of size ˜0.25 μm, dispersed in injectable gel

FIG. 3 represents the process steps involved in the preparation of drug loaded microparticles (˜0.25 μm) dispersed in injectable gel

FIG. 4 represents the drawing depicting the preparation of free drug loaded in microparticles of size ˜3 μm, dispersed in injectable gel

FIG. 5 represents the process steps involved in the preparation of drug loaded microparticles (˜3 μm) dispersed in injectable gel

FIG. 6 represents the drawing depicting the preparation of free drug loaded in microparticles of size 100-500 μm, dispersed in injectable gel

FIG. 7 represents the process steps involved in the preparation of drug loaded microparticles (100-500 μm) dispersed in injectable gel

FIG. 8A through 8F represents the size characterization of 0.25 μm particles, wherein FIG. 8A-C shows the size by intensity distribution, size by volume distribution and size by number distribution respectively, FIG. 8D shows the zeta potential distribution using DLS, FIG. 8E-F shows the morphological characteristics of the particles by SEM imaging.

FIG. 9A through 9F represents the size characterization of 3-5 μm sized particles, where FIG. 9A-C shows the size by intensity, volume and number distribution respectively, FIG. 9D shows the zeta potential distribution using DLS and FIG. 9E-F shows the morphological characteristics using FESEM.

FIG. 10A through 10F represents the morphological characteristics of 100-500 μm sized particles (loaded with BCNU) confirming its size, wherein FIG. 10A-C optical microscopy and FIG. 10D-F FESEM imaging is shown.

FIG. 11A through 11F represents the morphological characteristics of 100-500 μm sized microparticles loaded with Temozolomide (TMZ) using SEM imaging.

FIG. 12A through 12F represents the size analysis of BCNU released from microparticles of size 1-3 μm and its interaction with outer gel layer of PEG or its block copolymers. Size of (A) PEG 400-BCNU nanocomplexes, (B) PEG 6 kDa-BCNU nanocomplexes, (C) PEG 20 kDa-BCNU nanocomplexes, (D) Pluronic-BCNU nanocomplexes, (E) Kolliphor-BCNU nanocomplexes and (F) Soluplus-BCNU nanocomplexes.

FIG. 13A through 13F represents the size analysis of Piperlongumine (PL) released from microparticles of size 1-3 μm and its interaction with outer gel layer of PEG or its block copolymers. Size of (A) PEG 400-PL nanocomplexes, (B) PEG 6 kDa-PL nanocomplexes, (C) PEG 20 kDa-PL nanocomplexes, (D) Pluronic-PL nanocomplexes, (E) Kolliphor-PL nanocomplexes and (F) Soluplus-PL nanocomplexes.

FIG. 14A through 14C represents size characterization of in situ formed (A) Poloxamer-BCNU nanocomplexes, (B) Poloxamer-PL nanocomplexes and (C) Poloxamer-TMZ nanocomplexes after release of respective drug BCNU, Piperlongumine (PL) and Temozolomide (TMZ) from microparticles of size 1-3 μm (for BCNU and PL) and 100-500 μm for TMZ and their interaction with outer Pluronic gel layer.

FIG. 15A through 15E represents the size characterization of in situ formed nanocomplexes after release of drug from microparticles of size 100-500 μm and interaction with outer polymer gel layer of different molecular weights of PEG to form <100 nm sized FIG. 15A through 15D represents PEG 400 nanocomplexes formed with drugs TMZ (Temozolomide), 5-FU (5-Flourouracil), Dox (Doxorubicin) and Gemcitabine (Gem) and FIG. 15E through 15H represents PEG 6 kDa nanocomplexes formed with TMZ, 5-FU, Dox and Gem.

FIG. 16A through 16E represents the size characterization of in situ formed nanocomplexes after release of drug from microparticles of size 100-500 μm and interaction with outer polymer gel layer of different molecular weights of PEG or its block copolymers to form <100 nm sized FIG. 16A through 16D represents PEG 20 kDa nanocomplexes formed with drugs TMZ (Temozolomide), 5-FU (5-Flourouracil), Dox (Doxorubicin) and Gemcitabine (Gem) and FIG. 15E through 15H represents Pluronic nanocomplexes formed with TMZ, 5-FU, Dox and Gem.

FIG. 17A through 17E represents the size characterization of in situ formed nanocomplexes after release of drug from microparticles of size 100-500 μm and interaction with outer polymer gel layer of different molecular weights of PEG or its block copolymers to form <100 nm sized FIG. 17A through 17D represents Kolliphor nanocomplexes formed with drugs TMZ (Temozolomide), 5-FU (5-Flourouracil), Dox (Doxorubicin) and Gemcitabine (Gem) and FIG. 17E through 17H represents Soluplus nanocomplexes formed with TMZ, 5-FU, Dox and Gem.

FIG. 18A through 18F represents the size analysis of Paclitaxel (PTX) released from microparticles of size 100-500 μm and its interaction with outer gel layer of PEG or its block copolymers. Size of (A) PEG 400-PTX nanocomplexes, (B) PEG 6 kDa-PTX nanocomplexes, (C) PEG 20 kDa-PTX nanocomplexes, (D) Pluronic-PTX nanocomplexes, (E) Kolliphor-PTX nanocomplexes and (F) Soluplus-PTX nanocomplexes.

FIG. 19A through 19J shows the size and drug quantification of PEG-BCNU nanocomplexes (BCNU-NC-33), where FIG. 19(A-H) shows the morphology and size depiction of BCNU-NC-33 using TEM imaging, FIG. 191 shows the peaks exhibited by BCNU-NC-33, which are intermediatory compared to the peaks exhibited by free BCNU, PEG, using Raman spectroscopy and FIG. 19J shows the chromatogram of drug detected at 8.5 minutes using HPLC.

FIG. 20A through FIG. 20G represents the diffusion of ICG (Indocyanine green) loaded polymer nanocomplexes (ICG-NC-33) and ICG loaded 0.25 μm particles without gel coating, imaged over 4 hours using Near infrared imaging (NIR) in ex vivo goat brain phantom, where FIG. 20A shows the photograph and FIG. 20 (B-G) shows the NIR images at time points from 10^{th} minute to 5^{th} hour.

FIG. 21A through FIG. 21F represents diffusion of Free Iodine, Iodine loaded <100 nm nanocomplexes (Iodine-NC-33), Iodine-0.25 μm particles without gel coating, Iodine-0.25 μm particles coated with gel, Iodine-3 μm particles without gel coating and Iodine-3 μm particles coated with gel in an ex-vivo goat brain phantom imaged over 4 hours using CT.

FIG. 22A through 22D represents the in vitro cytotoxicity assay of free BCNU, BCNU-NC-33, BCNU-0.25 μm particles coated with gel and BCNU-3 μm particles coated with gel and free PL, PL-NC-33, PL-0.25 μm particles coated with gel and PL-3 μm particles coated with gel on monolayer C6 cells and T98G cells.

FIG. 23 shows Percentage of concentration of drug in in vivo rat brain, post injection of Free BCNU, BCNU-NC-33, BCNU-0.25 μm particles coated with gel and BCNU-3 μm particles coated with gel.

FIG. 24 (A-E) shows in vivo antitumor study of free BCNU, BCNU-0.25 μm particles coated with gel and BCNU-3 μm particles coated with gel in orthotopic tumor model in rat brain.

### DETAILED DESCRIPTION OF THE INVENTION

While the invention has been disclosed with reference to certain embodiments, it will be understood by those skilled in the art that various changes may be made, and equivalents may be substituted without departing from the scope of the invention. In addition, many modifications may be made to adapt to a particular situation or material to the teachings of the invention without departing from its scope.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein.

As used herein, the term “aqueous phase” refers to water or a solution in water.

As used herein “pharmaceutical acceptable salt” refers to inorganic and organic salt or salts of drug. These are well known to a skilled person.

As used herein “drug-polymer nanocomplex” refers to “polymer-drug nanocomplex, “nanocomplex”, or “nanodrug complexes”.

As used herein microstructure refers to microparticles of size ranging from 0.25-1000 microns.

As used herein an injectable microparticle system refers to as “injectable microparticle-gel system”, “a composite polymeric microparticle-gel system”, “single microparticle system”, “a drug-delivery system”, an injectable gel microstructure”, or “a composite drug-polymer nanocomplex”.

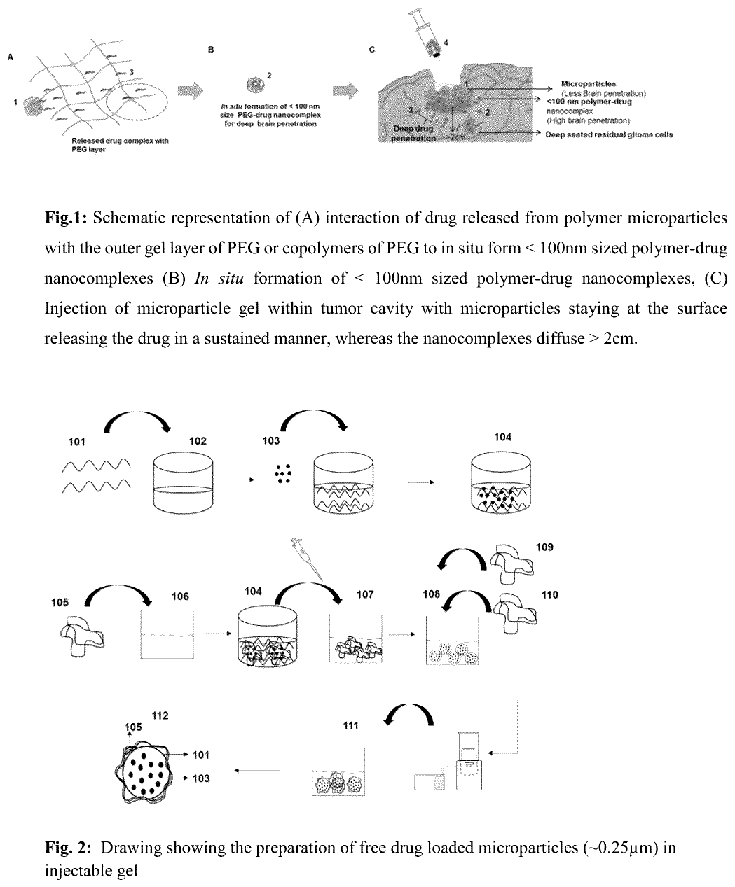

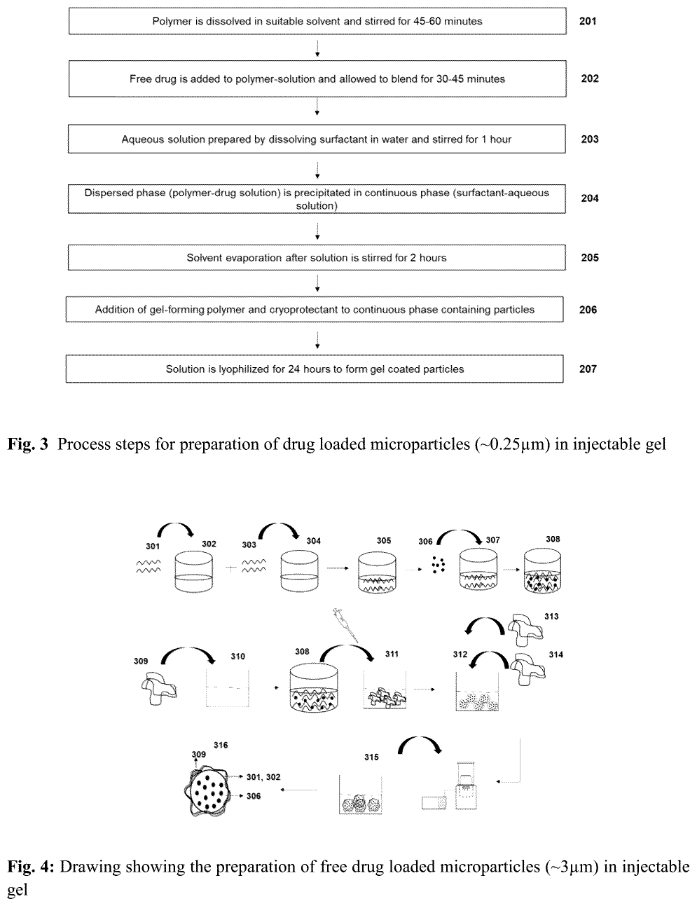

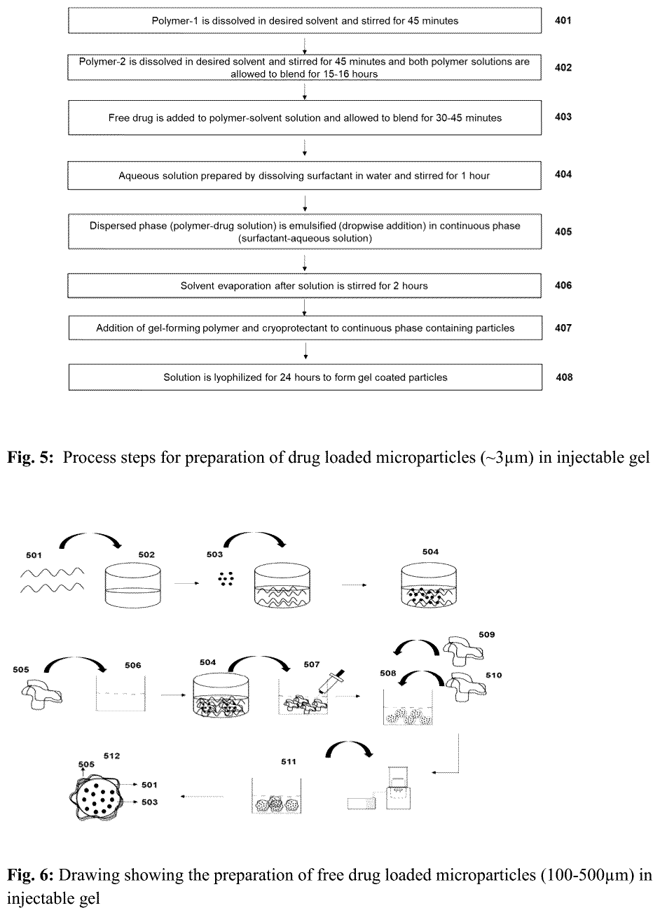

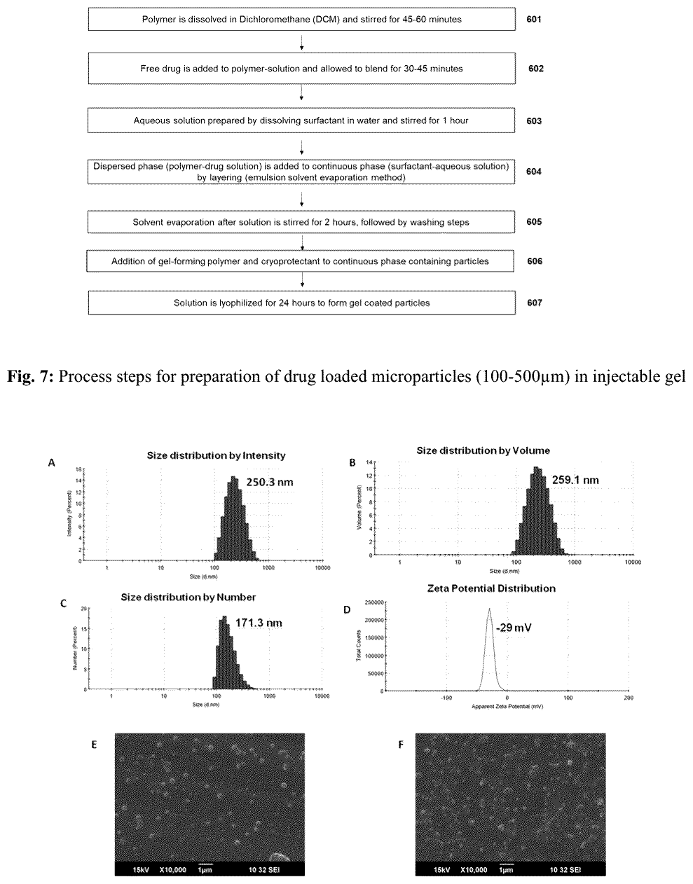

The present invention addresses the challenges in prior art by providing a novel innovative system wherein the drug-loaded microparticles can facilitate the release of drug-polymer nanocomplexes formed in situ by the interaction of the released drug with the outer polymer gel layer to form drug-polymer nanocomplexes of size <100 nm with larger tissue penetration of drug for >2 cm, while the injectable polymeric microparticle. The present invention achieves two keys benefits: a) sustained, prolonged release of chemo drug locally in the brain for 15-30 days as illustrated in FIG. 1A-C with b) enhanced brain-tissue penetration of the released drug-polymer nanocomplex for >2 cm which is critically required to treat deep seated or diffused brain tumors. Thus, innovative method and product of the present invention solve two critical issues of deep tissue brain drug delivery and sustained release up to 30 days in brain using a single microparticle system.

The deep brain-penetration of drug is a critical requirement for stopping recurrence of tumor due to residual tumor cells. The unique microparticle gel system is useful for other drug releasing applications requiring significant tissue penetration.

FIG. 1A-C depicts the method of in situ formation of drug-polymer nanocomplexes of size <100 nm, wherein the drug, 3, released by the microparticles, 1, interacts with the outer layer of PEG/Poloxamer/Soluplus®/Kolliphor/Pluronic or any block copolymers of PEG, leading to the in situ formation of drug-polymer nanocomplexes of size <100 nm 2. While the microparticles may remain in the injection site, the drug will be released due to diffusion or degradation of carrier microparticles. The released drug interacts with the outer polymer gel layer to form drug-polymer nanocomplexes, that will penetrate through the brain tissue and deliver free drug, 3, for >2 cm as depicted in FIG. 1C. The size of the drug-polymer nanocomplexes of <100 nm is less than the average pore size of the brain tissue (˜200 nm). The primary requirement of >2 cm drug penetration is facilitated by the inherent property of the in situ formed drug-polymer nanocomplexes.

In an embodiment, the present invention provides an injectable microparticle system comprising drug-loaded polymeric microparticles, wherein the drug-loaded polymeric microparticles are coated with an outer polymer gel layer enabling in-situ formation and releasing drug-polymer nanocomplexes of size <100 nm.

In an embodiment, the present invention provides the injectable microparticle system wherein drug-loaded polymeric microparticle is made of biodegradable and biocompatible polymer.

In an embodiment, the present invention provides the injectable microparticle system wherein the biodegradable and biocompatible polymer is selected from Poly lactic-co-glycolic acid (PLGA), Poly lactic acid (PLA), Polyvinyl alcohol (PVA), Poly caprolactam (PCL), or combination thereof.

In an embodiment, the present invention provides the injectable microparticle system wherein the size of drug-loaded polymeric microparticle is in a range of 0.25-1000 microns.

In an embodiment, the present invention provides the injectable microparticle system wherein a gel-forming polymer of outer polymer gel layer is selected from Polyethylene glycol having molecular weight ranging from 300-40000 Da, Poloxamer, Polyoxyl 15 hydroxystearate, Polyoxyl 35 Castor oil, Polysiloxane, Polysorbate 20, Polysorbate 80, Pluronic® (block copolymer of polyethylene oxide and polypropylene oxide), Soluplus® (graft copolymer of polyethylene glycol, polyvinylcaprolactam and polyvinylacetate), Kolliphor® (mixture of castor oil and ethylene oxide), or Polyvinyl alcohol.

In an embodiment, the present invention provides the injectable microparticle system wherein the ratio of polymer of the drug-loaded polymeric microparticles to gel-forming polymer is in a range of 1:0.1 to 1:50 w/w %.

In an embodiment, the present invention provides the injectable microparticle system wherein the drug loading within the polymeric microparticles ranges from 1% to 50% w/w.

In an embodiment, the present invention provides the injectable microparticle system wherein the microparticles are loaded with one or more chemotherapeutic drugs.

In an embodiment, the present invention provides the injectable microparticle system wherein the drug in the drug-loaded microparticles is selected from temozolomide, carmustine (BCNU), lomustine (CCNU), piperlongumine (PL), paclitaxel, cetuximab, irinotecan, everolimus, carboplatin, platinums, etoposide, methotrexate, Ara-c, pemetrexed, thiotepa, docetaxel, 5-flurouracil (5FU), 6-thioguanine (6TG), cisplatin, topotecan, bevacizumab, gemcitabine, doxorubicin, D-actinomycin, epirubicin, procarbazine, vincristine, tyrosine kinase inhibitors, kinase inhibitors, photodynamic therapy drugs, mTHPC, porphyrin, staurosporine, midostaurin, therapeutic proteins, GMCSF (Granulocyte-macrophage colony-stimulating factor), BDNF (Brain-Derived Neutrotrophic Factor), GCSF (Granulocyte colony-stimulating factor), MCSF (macrophage colony-stimulating factor), PEGylated G-CSF, PEGylated GM-CSF and pharmaceutically acceptable salt, acid, or derivative thereof.

In an embodiment, the present invention provides the injectable microparticle system wherein the drug loaded microparticles are in the form of lyophilized or freeze-dried powder.

In an embodiment, the present invention provides the injectable microparticle system wherein the drug-polymer nanocomplexes penetrate the brain for >2 cm.

In an embodiment, the present invention provides the injectable microparticle system wherein the drug from the drug-polymer nanocomplexes is released in a sustained manner for a period of 15-30 days.

In another embodiment, the present invention provides a method of preparing the injectable microparticle system comprising steps of:
(a) preparing a polymeric solution or a blend of polymeric solution by dissolving a polymer in an organic solvent;
(b) dissolving 1-50% wt/wt of a drug in the polymeric solution of step (a) to form a polymer-drug solution;
(c) forming a surfactant-aqueous solution by dissolving 0.1-50% w/v of a surfactant in water and stirring for 30 to 60 minutes;
(d) adding dropwise or directly injecting the polymer-drug solution of step (b) into the surfactant-aqueous solution of step (c) by stirring to form a micro-emulsion;
(e) evaporating the organic solvent from the micro-emulsion to obtain an aqueous phase having drug-loaded polymeric microparticles;
(f) optionally washing the aqueous phase of step (e) with 1% to 5% of polyvinyl alcohol (PVA), and then with deionized water by centrifugation or tangential flow filtration;
(g) coating the drug-loaded microparticle of step (e) or (f) by adding 0.1-50% w/v of a gel-forming polymer and 1 to 50% w/v of cryoprotectants in the aqueous phase and homogenizing;
(h) lyophilizing the homogenizing phase of step (g) to form drug-loaded polymeric microparticles powder coated and dispersed in gel forming polymer; and
(i) packing and sealing the lyophilized powder of step (h) in sterile condition.

In an embodiment, the present invention provides the method of preparing the injectable microparticle system wherein the blend of step (a) is prepared from the same polymer or two or three different polymers.

In an embodiment, the present invention provides the method of preparing the injectable microparticle system wherein the process optionally comprises a high pressure homogenization step to form the micro-emulsion at step (d).

In an embodiment, the present invention provides the method of preparing the injectable microparticle system wherein the organic solvent is selected from dichloromethane (DCM), acetone, 1,4-dioxane, chloroform, acetonitrile, dimethylformamide, ethyl acetate, methanol, ethanol, water, tetrahydrofuran, carbon tetrachloride, benzene, toluene, cyclohexanone, 2-nitropropane, or combination thereof.

In an embodiment, the present invention provides the method of preparing the injectable microparticle system wherein the polymer in step (a) is selected Poly lactic-co-glycolic acid (PLGA), Poly lactic acid (PLA), Polyvinyl alcohol (PVA), Poly caprolactam (PCL), or combination thereof.

In an embodiment, the present invention provides the method of preparing the injectable microparticle system wherein the drug is selected from temozolomide, carmustine (BCNU), lomustine (CCNU), piperlongumine (PL), paclitaxel, cetuximab, irinotecan, everolimus, carboplatin, platinums, etoposide, methotrexate, Ara-c, pemetrexed, thiotepa, docetaxel, 5-flurouracil (5FU), 6-thioguanine (6TG), cisplatin, topotecan, bevacizumab, gemcitabine, doxorubicin, D-actinomycin, epirubicin, procarbazine, vincristine, tyrosine kinase inhibitors, kinase inhibitors, photodynamic therapy drugs, mTHPC, porphyrin, staurosporine, midostaurin, therapeutic proteins, GMCSF (Granulocyte-macrophage colony-stimulating factor), BDNF (Brain-Derived Neutrotrophic Factor), GCSF (Granulocyte colony-stimulating factor), MCSF (macrophage colony-stimulating factor), PEGylated G-CSF, PEGylated GM-CSF and pharmaceutically acceptable salt, acid, or derivative thereof.

In an embodiment, the present invention provides the method of preparing the injectable microparticle system wherein drug loading within the polymeric microparticles range from 1% to 50% w/w to polymer of polymeric microparticles.

In an embodiment, the present invention provides the method of preparing the injectable microparticle system wherein the addition of surfactant in the water ranges from 0.1% to 50% w/v.

In an embodiment, the present invention provides the method of preparing the injectable microparticle system wherein the gel-forming polymer is selected from Polyethylene glycol having molecular weight ranging from 300-40000 Da, Poloxamer, Polyoxyl 15 hydroxystearate, Polyoxyl 35 Castor oil, Polysiloxane, Polysorbate 20, Polysorbate 80, Pluronic® (block copolymer of polyethylene oxide and polypropylene oxide), Soluplus® (graft copolymer of polyethylene glycol, polyvinylcaprolactam and polyvinylacetate), Kolliphor® (mixture of castor oil and ethylene oxide), or Polyvinyl alcohol.

In an embodiment, the present invention provides the method of preparing the injectable microparticle system wherein the addition of gel-forming polymer to the aqueous phase having drug-loaded polymeric microparticles in a range of 0.1% to 50% w/v.

In an embodiment, the present invention provides the method of preparing the injectable microparticle system wherein the cryoprotectant is selected from Polyethylene glycol (PEG) having molecular weight ranging from 300-40000 Da, Propylene glycol, Polyvinylpyrrolidone (PVP), Polyvinyl alcohol (PVA), Glycerol, 2-methyl-2, 4-pentanediol (MPD), Sucrose, Glucose, Fructose, Trehalose, Mannitol, Proline, Sorbitol, Dextran, Poloxamer or Pluronic® (block copolymer of polyethylene oxide and polypropylene oxide).

In an embodiment, the present invention provides the method of preparing the injectable microparticle system wherein the addition of cryoprotectant to the aqueous phase range from 1% to 50% w/v.

In an embodiment, the present invention provides the injectable microparticle system releasing polymer-drug nanocomplexes of size smaller than 100 nm for the purpose of intracranial drug delivery, wherein the unique design achieve two key benefits: a) sustained, prolonged release of chemo drug locally in the brain for 15-30 days and b) enhanced brain-tissue penetration of the released polymer-drug nanocomplex for >2 cm which is critically required to treat deep seated or diffused brain tumors.

In an embodiment, the present invention provides the use of injectable microparticle system in the treatment of brain tumor and other cancers.

In an embodiment, the present invention provides a method of treating brain tumor by introducing the injectable microparticle system into the brain tumor or tumor resected cavity or near to the tumor region in healthy brain tissue.

In an embodiment, the present invention provides the use of injectable microparticle system in combination with surgery, radiation therapy, photodynamic therapy, chemotherapy, immunotherapy, ultrasound therapy, radio-frequency ablation, tumor treating field therapy, cancer-vaccines or combinations thereof.

In one embodiment, the preparation of free drug 103 loaded microparticles of size ˜0.25 μm dispersed in gel is described (FIG. 2, 3). Initially, polymer-1 101 is dissolved in acetone and allowed to blend for 45-60 minutes as described in step 201. Step 202 describes the addition of free drug 103 to polymer-solution and stirred for 30-45 minutes. An aqueous solution is prepared by adding surfactant 105 to water 106 and stirring for 1 hour (step 203). Through drop-wise addition, the polymer-drug solution 104 is added to surfactant-aqueous solution 107 (step 204) and solvent is evaporated by stirring the solution for 2 hours (step 205). To the aqueous phase containing formed particles, gel forming polymer 109 and cryoprotectant 110 are added and allowed to blend (step 206). The microparticle containing solution 111 is freezed and lyophilized to form gel coated particles 112 (step 207).

In one embodiment, the polymer 101 and drug-polymer blend 103 can be dissolved in suitable solvents like dichloromethane (DCM), acetone, 1,4-Dioxane, chloroform, acetonitrile, dimethylformamide, ethyl acetate, methanol, ethanol, water, tetrahydrofuran, carbon tetrachloride, benzene, toluene, cyclohexanone, 2-nitropropane, or combination thereof.

In one embodiment, the preparation of free drug 306 loaded microparticles of size ˜3 μm dispersed in gel is described (FIG. 4, 5). Initially, polymer-1 301 is dissolved in suitable solvent 302 and allowed to blend for 45-60 minutes as described in step 401. Polymer-2 303 is dissolved in suitable solvent 304 and stirred for 45 minutes following which both the polymer solutions are allowed to blend for 15-16 hours (step 402). Step 403 describes the addition of free drug 306 to polymer blend-solution and stirred for 30-45 minutes. An aqueous solution is prepared by adding surfactant 309 to water 310 and stirring for 1 hour (step 404). Through drop-wise addition, the polymer-drug solution 308 is added to surfactant-aqueous solution 311 (step 405) and solvent is evaporated by stirring the solution for 2 hours (step 406). To the aqueous phase containing microparticles, gel forming polymer 313 and cryoprotectant 314 are added and dissolved (step 407). The microparticle containing solution 315 is freezed and lyophilized to form gel coated microparticles 316 (step 408).

In one embodiment, the preparation of free drug 503 loaded microparticles of size 100-500 μm dispersed in gel is described (FIG. 6, 7). Initially, polymer 501 is dissolved in acetone and allowed to blend for 45-60 minutes as described in step 601. Step 602 describes the addition of free drug 503 to polymer-solution and stirred for 30-45 minutes. An aqueous solution is prepared by adding surfactant 505 to water 506 and stirring for 1 hour (step 603). Through drop-wise addition, the polymer-drug solution 504 is added to surfactant-aqueous solution 507 using layering technique (step 604) and the solvent is evaporated by stirring the solution for 2 hours (step 605). To the aqueous phase containing microparticles 508, gel forming polymer 509 and cryoprotectant 510 are added and dissolved (step 606). The microparticle containing solution 511 is freezed and lyophilized to form gel coated microparticles 512 (step 607).

The polymer forming the microstructure is introduced by micro-emulsion or high-pressure homogenization or microfluidics. It is to be understood that the suitable materials for forming the microstructure are formed under prescribed conditions.

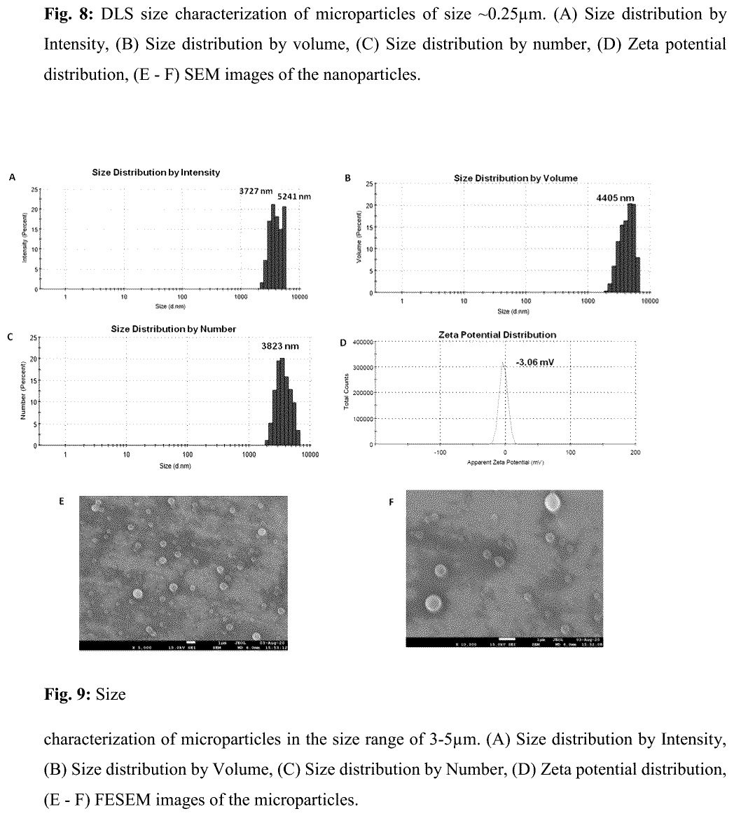

With the method of preparation of microparticles of size ˜0.25 μm as exemplified in Example 1, and depicted in FIG. 8A-F. The particle size analyzed using Dynamic light scattering (DLS) showed a size by intensity distribution of 0.250 μm (250.3 nm) (FIG. 8A), size by volume distribution of 0.259 μm (259.1 nm) (FIG. 8B) and size by number distribution of 0.171 μm (171.3 nm) (FIG. 8C) and zeta potential of −29 mV (FIG. 8D). FIG. 8E-F shows the Scanning Electron Microscopy (SEM) images of the microparticles.

With the method of preparation of microparticles of size ˜3-5 μm as exemplified in Example 2, and depicted in FIG. 9A-F. The particle size analyzed using DLS showed a size by intensity distribution of 3.727-5.241 μm (3727-5241 nm) (FIG. 9A), size by volume distribution of 4.405 μm (4405 nm) (FIG. 9B) and size by number distribution of 3.823 μm (3823 nm) (FIG. 9C) and zeta potential of −3.06 mV (FIG. 9D). FIG. 9E-F shows the Field Emission Scanning Electron Microscopy (FESEM) images of the microparticles.

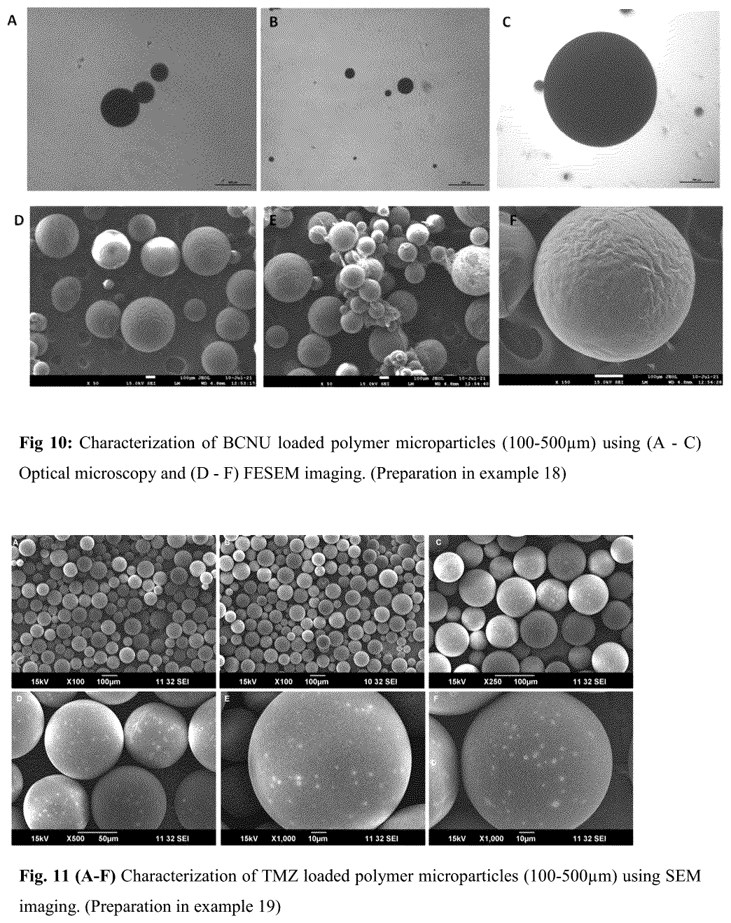

With the method of preparation of microparticles of size 100-500 μm loaded with the drug BCNU as exemplified in Example 16, and the particle size distribution is depicted using SEM imaging in FIG. 10A-F. Size characterization of microparticles (100-500 μm) loaded with Temozolomide (TMZ) drug (exemplified in Example 17) is depicted in FIG. 11A-F.

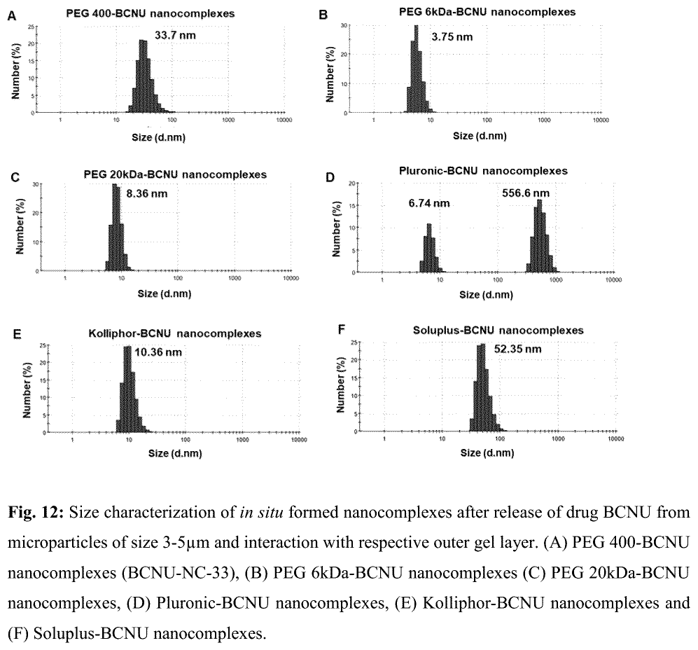

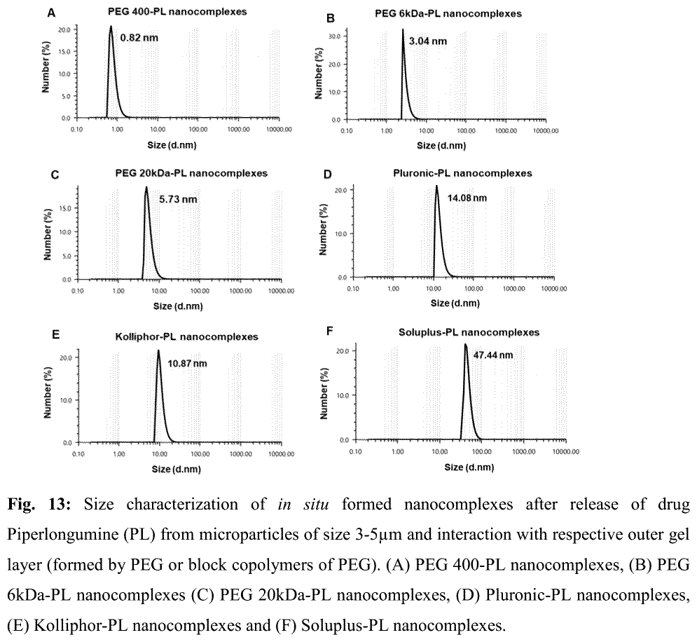

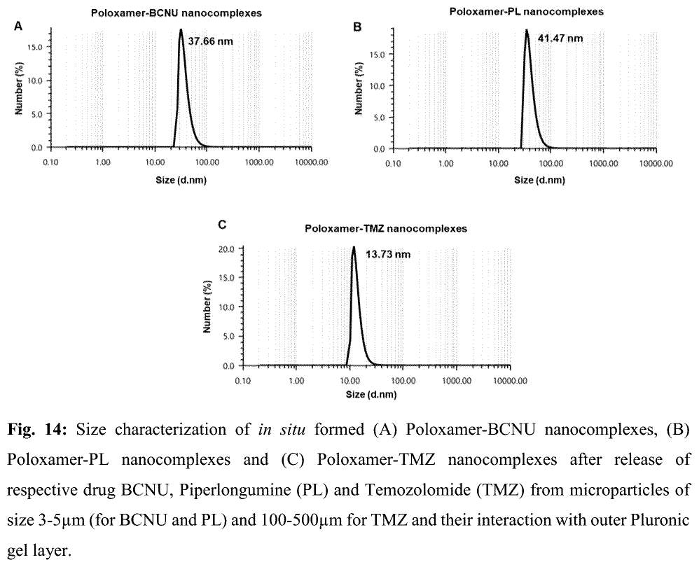

With the method of preparation of microparticles of size ˜3-5 μm (exemplified in Examples 3-8), the in situ formation of drug polymer-BCNU nancomplexes with different carrier polymers as gel coating over microparticles is characterized. FIG. 12A depicts the size analysis of PEG-400 BCNU nanocomplexes of 33.7 nm size (formed after BCNU release from the microparticles and its interaction with PEG 400 on the outer layer). FIG. 12B depicts the size analysis of PEG 6 kDa-BCNU nanocomplexes of 3.75 nm size (formed after BCNU release from the microparticles and its interaction with PEG 6 kDa on the outer layer). FIG. 12C depicts the size analysis of PEG-20 kDa-BCNU nanocomplexes of 8.36 nm size (formed after release from the microparticles and its interaction with PEG 20 kDa on the outer layer). FIG. 12D depicts the size analysis of Pluronic-BCNU nanocomplexes of 6.74 nm size (formed after BCNU release from the microparticles and its interaction with Pluronic on the outer layer). FIG. 12E depicts the size analysis of Kolliphor-BCNU nanocomplexes of 10.36 nm size (formed after BCNU release from the microparticles and its interaction with Kolliphor on the outer layer). FIG. 12F depicts the size analysis of Soluplus-BCNU nanocomplexes of 52.35 nm size (formed after BCNU release from the microparticles and its interaction with Soluplus on the outer layer) and FIG. 14A depicts the size analysis of Poloxamer-BCNU nanocomplexes of 37.66 nm size (formed after BCNU release from the microparticles and its interaction with Poloxamer on the outer layer).

With the method of preparation of microparticles of size ˜3-5 μm (exemplified in Examples 9-15), the in situ formation of drug polymer-Piperlongumine (PL) nancomplexes with different carrier polymers as gel coating over microparticles is characterized. FIG. 13A depicts the size analysis of PEG 400-PL nanocomplexes of 0.82 nm size (formed after PL release from the microparticles and its interaction with PEG 400 on the outer layer). FIG. 13B depicts the size analysis of PEG 6 kDa-PL nanocomplexes of 3.04 nm size (formed after PL release from the microparticles and its interaction with PEG 6 kDa on the outer layer). FIG. 13C depicts the size analysis of PEG 20 kDa-PL nanocomplexes of 5.73 nm size (formed after PL release from the microparticles and its interaction with PEG 20 kDa on the outer layer). FIG. 13D depicts the size analysis of Pluronic-PL nanocomplexes of 14.08 nm size (formed after PL release from the microparticles and its interaction with Pluronic on the outer layer). FIG. 13E depicts the size analysis of Kolliphor-PL nanocomplexes of 10.87 nm size (formed after PL release from the microparticles and its interaction with Kolliphor on the outer layer). FIG. 13F depicts the size analysis of Soluplus-PL nanocomplexes of 47.44 nm size (formed after PL release from the microparticles and its interaction with Soluplus on the outer layer) and FIG. 14B depicts the size analysis of Poloxamer-PL nanocomplexes of 41.47 nm size (formed after PL release from the microparticles and its interaction with Poloxamer on the outer layer).

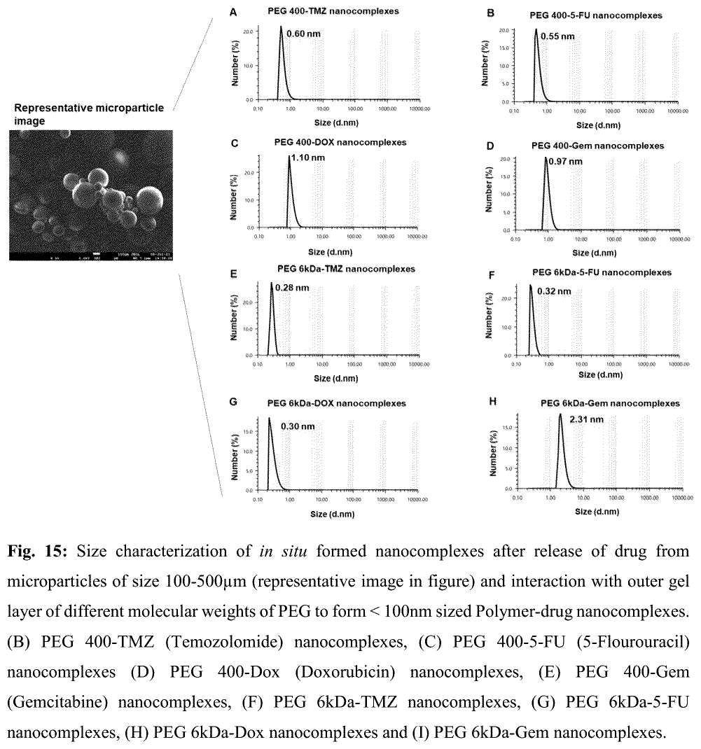

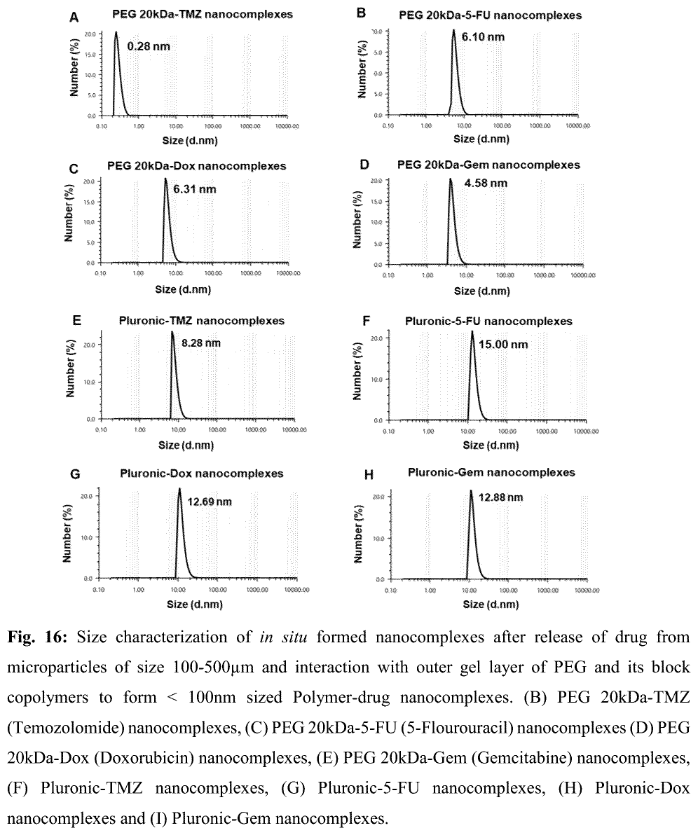

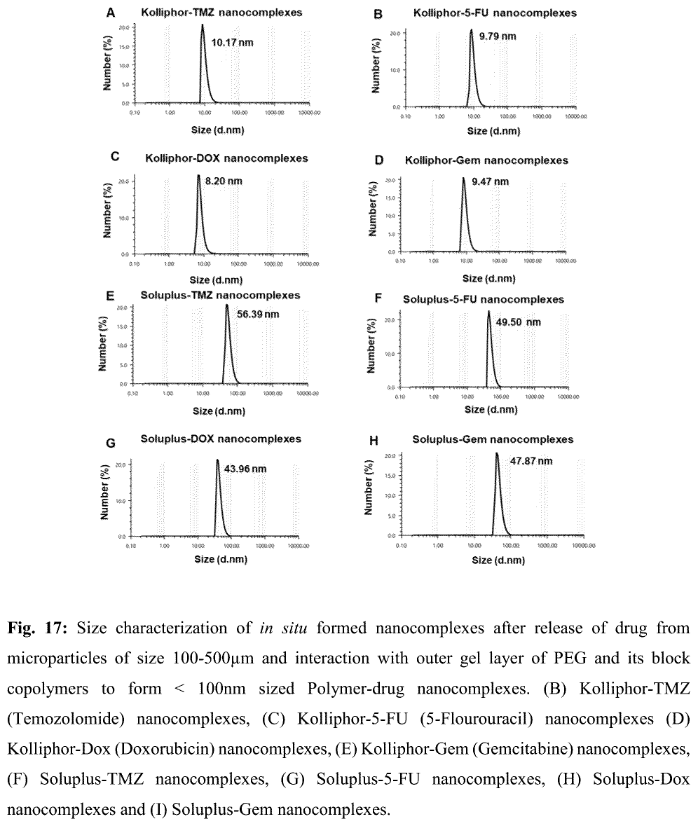

With the method of preparation of microparticles of size ˜100-500 μm, the in situ formation of drug polymer-Temozolomide (TMZ) nancomplexes with different carrier polymers as gel coating over microparticles is characterized (Examples 17-23). FIG. 15A depicts the size analysis of PEG 400-TMZ nanocomplexes of 0.60 nm size (formed after TMZ release from the microparticles and its interaction with PEG 400 on the outer layer). FIG. 15E depicts the size analysis of PEG 6 kDa-TMZ nanocomplexes of 0.28 nm size (formed after TMZ release from the microparticles and its interaction with PEG 6 kDa on the outer layer). FIG. 16A depicts the size analysis of PEG-20 kDa-TMZ nanocomplexes of 0.28 nm size (formed after TMZ release from the microparticles and its interaction with PEG 20 kDa on the outer layer). FIG. 16E depicts the size analysis of Pluronic-TMZ nanocomplexes of 8.28 nm size (formed after TMZ release from the microparticles and its interaction with Pluronic on the outer layer). FIG. 17A depicts the size analysis of Kolliphor-TMZ nanocomplexes of 10.17 nm size (formed after TMZ release from the microparticles and its interaction with Kolliphor on the outer layer). FIG. 17E depicts the size analysis of Soluplus-TMZ nanocomplexes of 56.39 nm size (formed after TMZ release from the microparticles and its interaction with Soluplus on the outer layer) and FIG. 14C depicts the size analysis of Poloxamer-TMZ nanocomplexes of 13.73 nm size (formed after TMZ release from the microparticles and its interaction with Poloxamer on the outer layer).

With the method of preparation of microparticles of size ˜100-500 μm, the in situ formation of drug polymer-5-Flourouracil (5-FU) nancomplexes with different carrier polymers as gel coating over microparticles is characterized (Examples 24-29). FIG. 15B depicts the size analysis of PEG 400-5-FU nanocomplexes of size 0.55 nm, FIG. 15F depicts the size analysis of PEG 6 kDa-5-FU nanocomplexes of size 0.32 nm, FIG. 16B depicts the size analysis of PEG 20 kDa-5-FU nanocomplexes of size 6.10 nm, FIG. 16F depicts the size analysis of Pluronic-5-FU nanocomplexes of size 15 nm, FIG. 17B depicts the size analysis of Kolliphor-5-FU nanocomplexes of 9.79 nm, FIG. 17F depicts the size analysis of Soluplus-5-FU nanocomplexes of 49.50 nm.

With the method of preparation of microparticles of size ˜100-500 μm, the in situ formation of drug polymer-Doxorubicin (Dox) nancomplexes with different carrier polymers as gel coating over microparticles is characterized (Examples 30-35). FIG. 15C depicts the size analysis of PEG 400-Dox nanocomplexes of size 1.10 nm, FIG. 15G depicts the size analysis of PEG 6 kDa-Dox nanocomplexes of size 0.30 nm, FIG. 16C depicts the size analysis of PEG 20 kDa-Dox nanocomplexes of size 6.31 nm, FIG. 16G depicts the size analysis of Pluronic-Dox nanocomplexes of size 12.69 nm, FIG. 17C depicts the size analysis of Kolliphor-Dox nanocomplexes of 8.20 nm, FIG. 17G depicts the size analysis of Soluplus-Dox nanocomplexes of 43.96 nm.

With the method of preparation of microparticles of size ˜100-500 μm, the in situ formation of drug polymer-Gemcitabine (Gem) nancomplexes with different carrier polymers as gel coating over microparticles is characterized (Examples 36-41). FIG. 15D depicts the size analysis of PEG 400-Gem nanocomplexes of size 0.97 nm, FIG. 15H depicts the size analysis of PEG 6 kDa-Gem nanocomplexes of size 2.31 nm, FIG. 16D depicts the size analysis of PEG 20 kDa-Gem nanocomplexes of size 4.58 nm, FIG. 16H depicts the size analysis of Pluronic-Gem nanocomplexes of size 12.88 nm, FIG. 17D depicts the size analysis of Kolliphor-Gem nanocomplexes of 9.47 nm, FIG. 17H depicts the size analysis of Soluplus-Gem nanocomplexes of 47.87 nm.

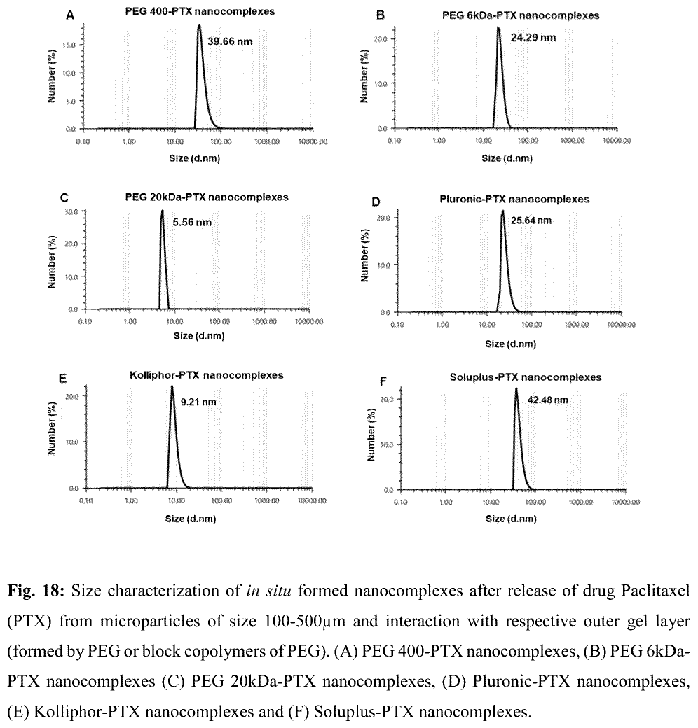

With the method of preparation of microparticles of size ˜100-500 μm, the in situ formation of drug polymer-Paclitaxel (PTX) nancomplexes with different carrier polymers as gel coating over microparticles is characterized (Examples 42-47). FIG. 18A depicts the size analysis of PEG 400-PTX nanocomplexes of size 39.66 nm, FIG. 18B depicts the size analysis of PEG 6 kDa-PTX nanocomplexes of size 24.29 nm, FIG. 18C depicts the size analysis of PEG 20 kDa-PTX nanocomplexes of size 5.56 nm, FIG. 18D depicts the size analysis of Pluronic-PTX nanocomplexes of size 25.64 nm, FIG. 18E depicts the size analysis of Kolliphor-PTX nanocomplexes of 9.21 nm, FIG. 18F depicts the size analysis of Soluplus-PTX nanocomplexes of 42.48 nm.

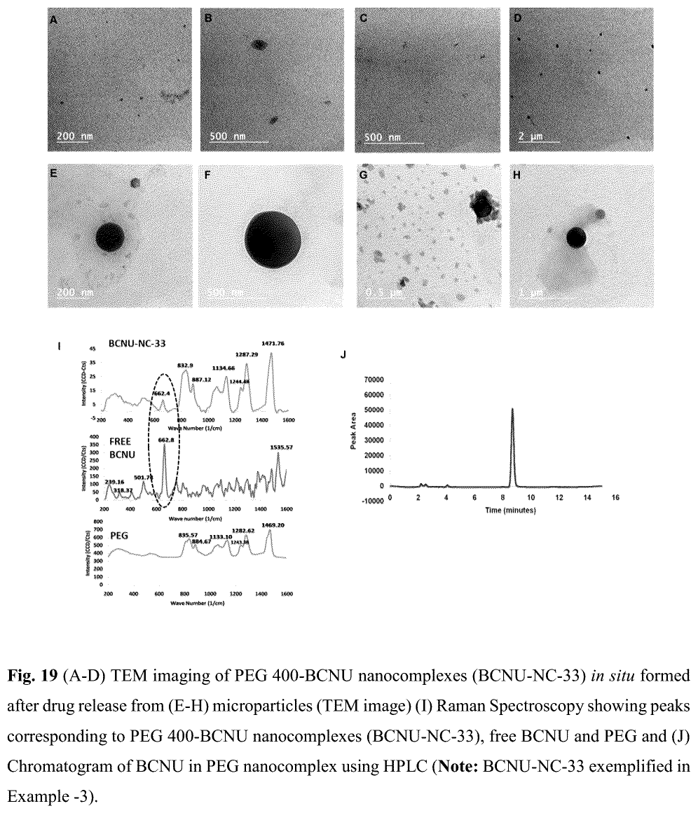

The morphology and size of drug-polymer nanocomplexes formed in situ (FIG. 19A-D) from microparticles (FIG. 19E-H) is depicted using Transmission Electron Microscopy (TEM) as exemplified in Examples 2 and 49. FIG. 191 shows the Raman spectroscopy confirming the presence of drug BCNU in BCNU-NC-33 nanocomplexes formed after the complexation of BCNU with the outer gel layer of PEG 400. Further confirmation of the presence of BCNU in BCNU-NC-33 nanocomplexes using High Pressure Liquid Chromatography (HPLC) as depicted in FIG. 19J.

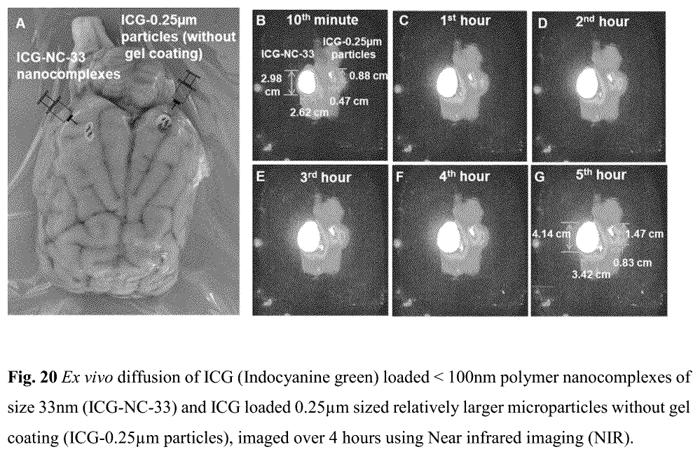

Ex vivo diffusion analysis of model drug Indocyanine Green (ICG) loaded microstructures were injected in left and right hemispheres of a goat brain phantom and imaged using Near Infrared Imaging (NIR) over 4 hours (FIG. 20 A-G). ICG loaded nanocomplexes (ICG-NC-33) showed >4 cm longitudinal diffusion and 3.4 cm lateral diffusion by 5^{th} hour, whereas ICG-0.25 μm particles without gel coating showed 1.47 cm longitudinal and 0.83 cm lateral diffusion by 5^{th} hour. ICG-NC-33 showed >3.5 cm increased in diffusion compared to ICG-0.25 μm particles without gel coating, depicting the importance of influence of size of particles for diffusion.

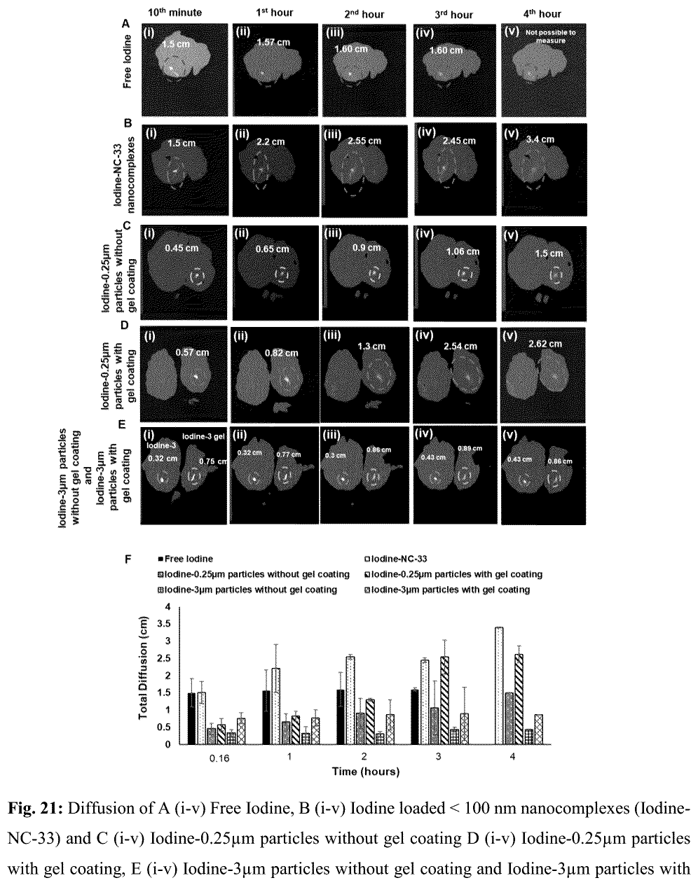

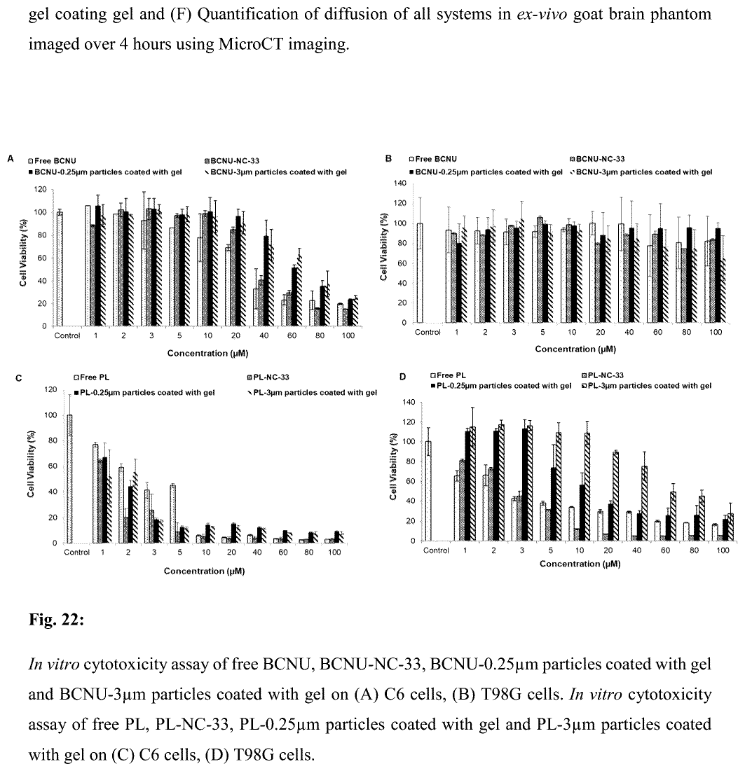

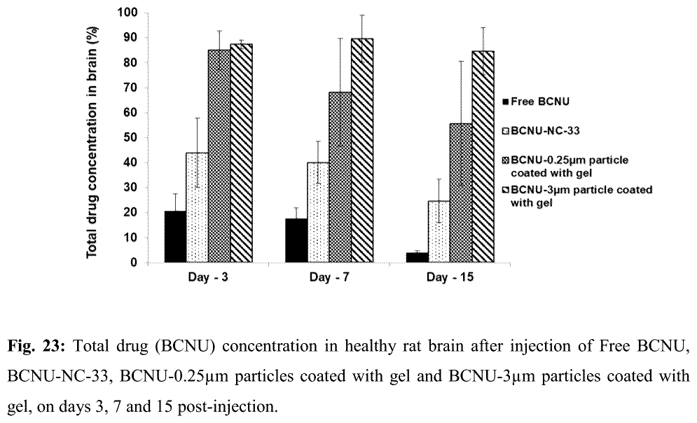

Contrast agent Iodine loaded microstructures are injected in left and right hemispheres of a goat brain phantom and imaged over 4 hours using MicroCT (FIG. 21 (A-E)). Free iodine diffused 1.6 cm by 3^{rd} hour, Iodine loaded nanocomplexes (Iodine-NC-33) showed >3.5 cm diffusion by 4^{th} hour, Iodine-0.25 μm particles without gel coating showed 1.5 cm, Iodine-0.25 μm particles coated with gel showed 2.62 cm, Iodine-3 μm particles without gel coating showed 0.43 cm and Iodine-3 μm particles coated with gel showed 0.86 cm by the 4^{th} hour. Iodine-NC-33 nanocomplexes also showed maximum diffusion of >3 cm amongst the systems tested.

Whereas, in the case of in vivo BCNU concentration in rat brain, free BCNU showed the least drug of 15% on 3^{rd} day which degraded to 3.9% on day-15 (FIG. 23). BCNU-NC-33 nanocomplexes showed 82.8% drug on day-3 which reduced to 24.6% on day-15. However, with size of microparticles in the larger size range, BCNU-0.25 μm particles with gel coating and BCNU-3 μm particles with gel coating showed 90.4%, 86.1% drug on day-3 and 55.6%, 73.6% drug on day-15 respectively. BCNU-3 μm particles coated with gel protected maximum amount of drug with ˜70% and ˜50% more retention compared to free BCNU and BCNU-NC-33 drug-polymer nanocomplexes respectively.

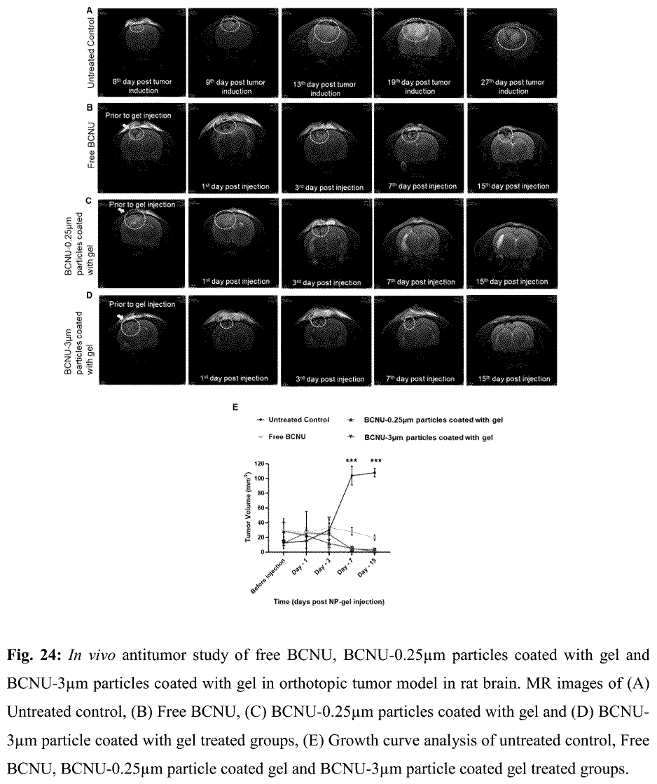

In vivo tumor growth curve showed a faster reduction in tumor with BCNU-0.25 μm particles coated with gel by 49.94%, 16.38% by day-1, to 98.99%, 95.46% by day-15 compared to untreated control and free BCNU (FIG. 24B) respectively. BCNU-3 μm particles coated with gel showed a decrease in tumor volume by 72.37%, 3.88% by day-1, to 96.92%, 86.05% by day-15 post injection (FIG. 24D). Faster reduction in tumor volume by BCNU-0.25 μm particles coated with gel was observed compared to BCNU-3 μm particles coated with gel, due to immediate release by smaller sized particles (FIG. 24E).

In various embodiments, the microstructure refers to microparticles of size ranging from 0.25-1000 microns. These particles can be loaded with MR or CT contrast agent, thus enabling them to be imaged using MRI or CT imaging.

The release of drug from this microstructure, thus forming drug-polymer nanocomplexes in situ, result in drug diffusion in lateral and vertical directions as demonstrated in FIG. 20 and FIG. 21.

In many embodiments, the amount of drug encapsulated within the microstructure may be modified. The change in encapsulation can be achieved by changing the concentration of polymer forming the microstructure, varying the molecular weight of the microstructure polymer, prior to addition of drug.

The release kinetics of the microstructure can be modified by changing the polymers used in forming the microstructure, its molecular weight. Different levels of drug released to the tissues, leading to different levels of cellular aberrations and cellular apoptosis.

In various embodiments, the microstructure is formed by the process of precipitation, micro-emulsion, solvent evaporation, high-pressure homogenization, microfluidics.

In various embodiments, the encapsulation of drug within the microstructure is achieved by precipitation, emulsion, solvent evaporation, or combinations thereof.

In some embodiments, the present invention provides controlled release of drug at two levels, one from the microstructure releasing the drug in a sustained manner into the surrounding gel material and second, from the nanocomplexes formed by the interaction drug with gel material that travel a distance of >2 cm in brain tissue to deliver drug immediately to treat cells present in diffuse regions.

Certain specific aspects and embodiments of the present application will be explained in greater detail with reference to the following examples, which are provided only for purposes of illustration and should not be construed as limiting the scope of the application in any manner. While particular aspects of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

### EXAMPLES

The following examples are for the purpose of illustration of the invention and are not intended in any way to limit the scope of the invention.

### Example 1: Development of Injectable Gel Microstructure Consisting of 0.25 μm PLGA Particle Loaded with BCNU Drug (50 wt. %) and Surface Coated with PEG 400 In Situ Releasing BCNU-PEG Nanocomplexes of Size <100 nm

In this example, preparation of microparticles of size ˜0.25 μm loaded with 50% BCNU loading is described (FIG. 2, 3). 70 mg PLGA (75:25, 20 kDa) is dissolved in 2 ml Acetone and allowed to stir at 500 rpm for 45 minutes to 1 hour. 0.5% w/v PEG (20 kDa) is dissolved in 10 ml MilliQ water at 700-800 rpm, for 1 hour. BCNU (once dissolved in suitable solvent) is added to PLGA solution and allowed to blend for 2 hours, following which it is precipitated in Pegylated aqueous solution. Following precipitation, solution is allowed to stir for 2-4 hours to evaporate the solvent. The gel-forming polymer (PEG 400) of 50% w/v and cryoprotectant (Pluronic) of 15% w/v are added to aqueous phase containing microparticles and allowed to dissolve (washing of particles using centrifugation or tangential flow filtration can be done as an optional step prior to adding gel-forming polymer). The precipitate is lyophilized for 24 hours to obtain the final product. Once the microparticle-gel is injected in the brain, the drug released from the dispersed PLGA particles will complex with the outer layer of PEG 400 (gel-forming material) to in situ form <100 nm sized PEG-drug nanocomplexes. Size characterization of particles is depicted in FIG. 8A-F.

### Example 2: Development of Injectable Gel Microstructure Consisting of 0.25 μm PLGA Particle Loaded with Temozolomide (TMZ) Drug (50 wt. %) and Surface Coated with PEG 400 In Situ Releasing TMZ-PEG Nanocomplexes of Size <100 nm

In this example, preparation of microparticles of size ˜0.25 μm loaded with 50% TMZ loading is described (FIG. 2, 3). 70 mg PLGA (75:25, 20 kDa) is dissolved in 2 ml Acetone and allowed to stir at 500 rpm for 45 minutes to 1 hour. 0.5% w/v PEG (20 kDa) is dissolved in 10 ml MilliQ water at 700-800 rpm, for 1 hour. Temozolomide (once dissolved in suitable solvent) is added to PLGA solution and allowed to blend for 2 hours, following which it is precipitated in Pegylated aqueous solution. Following precipitation, solution is allowed to stir for 2-4 hours to evaporate the solvent. The gel-forming polymer (PEG 400) of 50% w/v and cryoprotectant (Pluronic) of 15% w/v are added to aqueous phase containing microparticles and allowed to dissolve (washing of particles using centrifugation or tangential flow filtration can be done as an optional step prior to adding gel-forming polymer). The precipitate is lyophilized for 24 hours to obtain the final product. Once the microparticle-gel is injected in the brain, the drug released from the dispersed PLGA particles will complex with the outer layer of PEG 400 (gel-forming material) to in situ form <100 nm sized PEG-drug nanocomplexes.

### Example 3: Development of Injectable Gel Microstructure Consisting of 3-5 μm PLGA Particles Loaded with Drug BCNU (16.6 wt. %) and Surface Coated with PEG 400 In Situ Releasing PEG 400-BCNU Nanocomplexes of Size <100 nm

In this example, preparation of microparticles of size 3-5 μm loaded with 16.6% BCNU is described (FIG. 4, 5). 100 mg PLGA (75:25, 20 kDa) and 100 mg PLA (37 kDa) are dissolved in 2 ml Acetone and allowed to stir at 500 rpm for 45 minutes to 1 hour. 2% w/v PEG (20 kDa) is dissolved in 10 ml MilliQ water at 700-800 rpm, for 1 hour. BCNU (once dissolved in suitable solvent) is added to PLGA solution and allowed to blend for 2 hours, following which it is emulsified in PEG solution. Following emulsification, solution is allowed to stir for 2-4 hours to evaporate the solvent. The gel-forming polymer (PEG 400) of 50% w/v and cryoprotectant (Glucose) of 15% w/v are added to aqueous phase containing microparticles and allowed to dissolve (washing of particles using centrifugation or tangential flow filtration can be done as an optional step prior to adding gel-forming polymer). The emulsion is lyophilized for 24 hours to obtain the final product. Once the microparticle-gel is injected in the brain, the drug released from the dispersed PLGA particles will complex with the outer layer of PEG 400 (gel-forming material) to in situ form <100 nm sized PEG-drug nanocomplexes, BCNU-NC-33 (FIG. 12A). Size characterization of microparticles is depicted in FIG. 9A-F.

### Example 4: Development of Injectable Gel Microstructure Consisting of 3-5 μm PLGA Particles Loaded with Drug BCNU (16.6 wt. %) and Surface Coated with PEG 6 kDa In Situ Releasing PEG 6 kDa-BCNU Nanocomplexes of Size <100 nm

In this example, preparation of microparticles of size 3-5 μm loaded with 16.6% BCNU is described (FIG. 4, 5). 100 mg PLGA (75:25, 20 kDa) and 100 mg PLA (37 kDa) are dissolved in 2 ml Acetone and allowed to stir at 500 rpm for 45 minutes to 1 hour. 2% w/v PEG (20 kDa) is dissolved in 10 ml MilliQ water at 700-800 rpm, for 1 hour. BCNU (once dissolved in suitable solvent) is added to PLGA solution and allowed to blend for 2 hours, following which it is emulsified in PEG solution. Following emulsification, solution is allowed to stir for 2-4 hours to evaporate the solvent. The gel-forming polymer (PEG 6 kDa) of 25% w/v and cryoprotectant (Glucose) of 15% w/v are added to aqueous phase containing microparticles and allowed to dissolve (washing of particles using centrifugation or tangential flow filtration can be done as an optional step prior to adding gel-forming polymer). The emulsion is lyophilized for 24 hours to obtain the final product. Once the microparticle-gel is injected in brain, the drug released from the dispersed PLGA particles will complex with the outer layer of PEG 6 kDa (gel-forming material) to in situ form <100 nm sized PEG-drug nanocomplexes (FIG. 12B).

### Example 5: Development of Injectable Gel Microstructure Consisting of 3-5 μm PLGA Particles Loaded with Drug BCNU (16.6 wt. %) and Surface Coated with PEG 20 kDa In Situ Releasing PEG 20 kDa-BCNU Nanocomplexes of Size <100 nm

In this example, preparation of microparticles of size 3-5 μm loaded with 16.6% BCNU is described (FIG. 4, 5). 100 mg PLGA (75:25, 20 kDa) and 100 mg PLA (37 kDa) are dissolved in 2 ml Acetone and allowed to stir at 500 rpm for 45 minutes to 1 hour. 2% w/v PEG (20 kDa) is dissolved in 10 ml MilliQ water at 700-800 rpm, for 1 hour. BCNU (dissolved in suitable solvent) is added to PLGA solution and allowed to blend for 2 hours, following which it is emulsified in PEG solution. Following emulsification, solution is allowed to stir for 2-4 hours to evaporate the solvent. The gel-forming polymer (PEG 20 kDa) of 25% w/v and cryoprotectant (Glucose) of 15% w/v are added to aqueous phase containing microparticles and allowed to dissolve (washing of particles using centrifugation or tangential flow filtration can be done as an optional step prior to adding gel-forming polymer). The emulsion is lyophilized for 24 hours to obtain the final product. Once the microparticle-gel is injected in the brain, the drug released from the dispersed PLGA particles will complex with the outer layer of PEG 20 kDa (gel-forming material) to in situ form <100 nm sized PEG-drug nanocomplexes (FIG. 12C).

### Example 6: Development of Injectable Gel Microstructure Consisting of 3-5 μm PLGA Particles Loaded with Drug BCNU (16.6 wt. %) and Surface Coated with Poloxamer In Situ Releasing Poloxamer-BCNU Nanocomplexes of Size <100 nm

In this example, preparation of microparticles of size 3-5 μm loaded with 16.6% BCNU is described (FIG. 4, 5). 100 mg PLGA (75:25, 20 kDa) and 100 mg PLA (37 kDa) are dissolved in 2 ml Acetone and allowed to stir at 500 rpm for 45 minutes to 1 hour. 2% w/v PEG (20 kDa) is dissolved in 10 ml MilliQ water at 700-800 rpm, for 1 hour. BCNU (dissolved in suitable solvent) is added to PLGA solution and allowed to blend for 2 hours, following which it is emulsified in PEG solution. Following emulsification, solution is allowed to stir for 2-4 hours to evaporate the solvent. The gel-forming polymer (Poloxamer) of 25% w/v and cryoprotectant (Glucose) of 15% w/v are added to aqueous phase containing microparticles and allowed to dissolve (washing of particles using centrifugation or tangential flow filtration can be done as an optional step prior to adding gel-forming polymer). The emulsion is lyophilized for 24 hours to obtain the final product. Once the microparticle-gel is injected in brain, the drug released from the dispersed PLGA particles will complex with the outer layer of Poloxamer (gel-forming material) to in situ form <100 nm sized Poloxamer-drug nanocomplexes (FIG. 14A).

### Example 7: Development of Injectable Gel Microstructure Consisting of 3-5 μm PLGA Particles Loaded with Drug BCNU (16.6 wt. %) and Surface Coated with Pluronic In Situ Releasing Pluronic-BCNU Nanocomplexes of Size <100 nm

In this example, preparation of microparticles of size 3-5 μm loaded with 16.6% BCNU is described (FIG. 4, 5). 100 mg PLGA (75:25, 20 kDa) and 100 mg PLA (37 kDa) are dissolved in 2 ml Acetone and allowed to stir at 500 rpm for 45 minutes to 1 hour. 2% w/v PEG (20 kDa) is dissolved in 10 ml MilliQ water at 700-800 rpm, for 1 hour. BCNU (dissolved in suitable solvent) is added to PLGA solution and allowed to blend for 2 hours, following which it is emulsified in PEG solution. Following emulsification, solution is allowed to stir for 2-4 hours to evaporate the solvent. The gel-forming polymer (Pluronic) of 25% w/v and cryoprotectant (Glucose) of 15% w/v are added to aqueous phase containing microparticles and allowed to dissolve (washing of particles using centrifugation or tangential flow filtration can be done as an optional step prior to adding gel-forming polymer). The emulsion is lyophilized for 24 hours to obtain the final product. Once the microparticle-gel is injected in brain, the drug released from the dispersed PLGA particles will complex with the outer layer of Pluronic (gel-forming material) to in situ form <100 nm sized Pluronic-drug nanocomplexes (FIG. 12D).

### Example 8: Development of Injectable Gel Microstructure Consisting of 3-5 μm PLGA Particles Loaded with Drug BCNU (16.6 wt. %) and Surface Coated with Kolliphor In Situ Releasing Kolliphor-BCNU Nanocomplexes of Size <100 nm

In this example, preparation of microparticles of size 3-5 μm loaded with 16.6% BCNU is described (FIG. 4, 5). 100 mg PLGA (75:25, 20 kDa) and 100 mg PLA (37 kDa) are dissolved in 2 ml Acetone and allowed to stir at 500 rpm for 45 minutes to 1 hour. 2% w/v PEG (20 kDa) is dissolved in 10 ml MilliQ water at 700-800 rpm, for 1 hour. BCNU (dissolved in suitable solvent) is added to PLGA solution and allowed to blend for 2 hours, following which it is emulsified in PEG solution. Following emulsification, solution is allowed to stir for 2-4 hours to evaporate the solvent. The gel-forming polymer (Kolliphor) of 25% w/v and cryoprotectant (Glucose) of 15% w/v are added to aqueous phase containing microparticles and allowed to dissolve (washing of particles using centrifugation or tangential flow filtration can be done as an optional step prior to adding gel-forming polymer). The emulsion is lyophilized for 24 hours to obtain the final product. Once the microparticle-gel is injected in brain, the drug released from the dispersed PLGA particles will complex with the outer layer of Kolliphor (gel-forming material) to in situ form <100 nm sized Kolliphor-drug nanocomplexes (FIG. 12E).

### Example 9: Development of Injectable Gel Microstructure Consisting of 3-5 μm PLGA Particles Loaded with Drug BCNU (16.6 wt. %) and Surface Coated with Soluplus In Situ Releasing Soluplus-BCNU Nanocomplexes of Size <100 nm

In this example, preparation of microparticles of size 3-5 μm loaded with 16.6% BCNU is described (FIG. 4, 5). 100 mg PLGA (75:25, 20 kDa) and 100 mg PLA (37 kDa) are dissolved in 2 ml Acetone and allowed to stir at 500 rpm for 45 minutes to 1 hour. 2% w/v PEG (20 kDa) is dissolved in 10 ml MilliQ water at 700-800 rpm, for 1 hour. BCNU (dissolved in suitable solvent) is added to PLGA solution and allowed to blend for 2 hours, following which it is emulsified in PEG solution. Following emulsification, solution is allowed to stir for 2-4 hours to evaporate the solvent. The gel-forming polymer (Soluplus) of 25% w/v and cryoprotectant (Glucose) of 15% w/v are added to aqueous phase containing microparticles and allowed to dissolve (washing of particles using centrifugation or tangential flow filtration can be done as an optional step prior to adding gel-forming polymer). The emulsion is lyophilized for 24 hours to obtain the final product. Once the microparticle-gel is injected in brain, the drug released from the dispersed PLGA particles will complex with the outer layer of Soluplus (gel-forming material) to in situ form <100 nm sized Soluplus-drug nanocomplexes (FIG. 12F).

### Example 10: Development of Injectable Gel Microstructure Consisting of 3-5 μm PLGA Particles Loaded with Drug Temozolomode (TMZ) (10 wt. %) and Surface Coated with PEG 400 In Situ Releasing PEG 400-TMZ Nanocomplexes of Size <100 nm

In this example, preparation of microparticles of size 3-5 μm loaded with 10% Temozolomide (TMZ) is described (FIG. 4, 5). 100 mg PLGA (75:25, 20 kDa) and 100 mg PLA (37 kDa) are dissolved in 2 ml Acetone and allowed to stir at 500 rpm for 45 minutes to 1 hour. 2 wt. % PEG (20 kDa) is dissolved in 10 ml MilliQ water at 700-800 rpm, for 1 hour. TMZ (dissolved in suitable solvent) is added to PLGA solution and allowed to blend for 2 hours, following which it is emulsified in PEG solution. Following emulsification, solution is allowed to stir for 2-4 hours to evaporate the solvent. The gel-forming polymer (PEG 400) of 50% w/v and cryoprotectant (Glucose) of 15% w/v are added to aqueous phase containing microparticles and allowed to dissolve (washing of particles using centrifugation or tangential flow filtration can be done as an optional step prior to adding gel-forming polymer). The emulsion is lyophilized for 24 hours to obtain the final product. Once the microparticle-gel is injected in brain, the drug released from the dispersed PLGA particles will complex with the outer layer of PEG 400 (gel-forming material) to in situ form <100 nm sized PEG-drug nanocomplexes.

### Example 11: Development of Injectable Gel Microstructure Consisting of 3-5 μm PLGA Particles Loaded with Drug Piperlongumine (10 wt. %) and Surface Coated with PEG 400 In Situ Releasing PEG 400-PL (Piperlongumine) Nanocomplexes of Size <100 nm

In this example, preparation of microparticles of size 3-5 μm loaded with 10% Piperlongumine (PL) is described (FIG. 4, 5). 100 mg PLGA (75:25, 20 kDa) and 100 mg PLA (37 kDa) are dissolved in 2 ml Acetone and allowed to stir at 500 rpm for 45 minutes to 1 hour. 2% w/v PEG (20 kDa) is dissolved in 10 ml MilliQ water at 700-800 rpm, for 1 hour. PL (dissolved in suitable solvent) is added to PLGA solution and allowed to blend for 2 hours, following which it is emulsified in PEG solution. Following emulsification, solution is allowed to stir for 2-4 hours to evaporate the solvent. The gel-forming polymer (PEG 400) of 50% w/v and cryoprotectant (Glucose) of 15% w/v are added to aqueous phase containing microparticles and allowed to dissolve (washing of particles using centrifugation or tangential flow filtration can be done as an optional step prior to adding gel-forming polymer). The emulsion is lyophilized for 24 hours to obtain the final product. Once the microparticle-gel is injected in brain, the drug released from the dispersed PLGA particles will complex with the outer layer of PEG 400 (gel-forming material) to in situ form <100 nm sized PEG-drug nanocomplexes (FIG. 13A).

### Example 12: Development of Injectable Gel Microstructure Consisting of 3-5 μm PLGA Particles Loaded with Drug Piperlongumine (10 wt. %) and Surface Coated with PEG 6 kDa In Situ Releasing PEG 6 kDa-PL (Piperlongumine) Nanocomplexes of Size <100 nm

In this example, preparation of microparticles of size 3-5 μm loaded with 10% Piperlongumine (PL) is described (FIG. 4, 5). 100 mg PLGA (75:25, 20 kDa) and 100 mg PLA (37 kDa) are dissolved in 2 ml Acetone and allowed to stir at 500 rpm for 45 minutes to 1 hour. 2% w/v PEG (20 kDa) is dissolved in 10 ml MilliQ water at 700-800 rpm, for 1 hour. PL (dissolved in suitable solvent) is added to PLGA solution and allowed to blend for 2 hours, following which it is emulsified in PEG solution. Following emulsification, solution is allowed to stir for 2-4 hours to evaporate the solvent. The gel-forming polymer (PEG 6 kDa) of 25% w/v and cryoprotectant (Glucose) of 15% w/v are added to aqueous phase containing microparticles and allowed to dissolve (washing of particles using centrifugation or tangential flow filtration can be done as an optional step prior to adding gel-forming polymer). The emulsion is lyophilized for 24 hours to obtain the final product. Once the microparticle-gel is injected in brain, the drug released from the dispersed PLGA particles will complex with the outer layer of PEG 6 kDa (gel-forming material) to in situ form <100 nm sized PEG-drug nanocomplexes (FIG. 13B).

### Example 13: Development of Injectable Gel Microstructure Consisting of 3-5 μm PLGA Particles Loaded with Drug Piperlongumine (10 wt. %) and Surface Coated with PEG 20 kDa In Situ Releasing PEG 20 kDa-PL (Piperlongumine) Nanocomplexes of Size <100 nm

In this example, preparation of microparticles of size 3-5 μm loaded with 10% Piperlongumine (PL) is described (FIG. 4, 5). 100 mg PLGA (75:25, 20 kDa) and 100 mg PLA (37 kDa) are dissolved in 2 ml Acetone and allowed to stir at 500 rpm for 45 minutes to 1 hour. 2% w/v PEG (20 kDa) is dissolved in 10 ml MilliQ water at 700-800 rpm, for 1 hour. PL (dissolved in suitable solvent) is added to PLGA solution and allowed to blend for 2 hours, following which it is emulsified in PEG solution. Following emulsification, solution is allowed to stir for 2-4 hours to evaporate the solvent. The gel-forming polymer (PEG 20 kDa) of 25% w/v and cryoprotectant (Glucose) of 15% w/v are added to aqueous phase containing microparticles and allowed to dissolve (washing of particles using centrifugation or tangential flow filtration can be done as an optional step prior to adding gel-forming polymer). The emulsion is lyophilized for 24 hours to obtain the final product. Once the microparticle-gel is injected in brain, the drug released from the dispersed PLGA particles will complex with the outer layer of PEG 20 kDa (gel-forming material) to in situ form <100 nm sized PEG-drug nanocomplexes (FIG. 13C).

### Example 14: Development of Injectable Gel Microstructure Consisting of 3-5 μm PLGA Particles Loaded with Drug Piperlongumine (10 wt. %) and Surface Coated with Poloxamer In Situ Releasing Poloxamer-PL (Piperlongumine) Nanocomplexes of Size <100 nm

In this example, preparation of microparticles of size 3-5 μm loaded with 10% Piperlongumine (PL) is described (FIG. 4, 5). 100 mg PLGA (75:25, 20 kDa) and 100 mg PLA (37 kDa) are dissolved in 2 ml Acetone and allowed to stir at 500 rpm for 45 minutes to 1 hour. 2% w/v PEG (20 kDa) is dissolved in 10 ml MilliQ water at 700-800 rpm, for 1 hour. PL (dissolved in suitable solvent) is added to PLGA solution and allowed to blend for 2 hours, following which it is emulsified in PEG solution. Following emulsification, solution is allowed to stir for 2-4 hours to evaporate the solvent. The gel-forming polymer (Poloxamer) of 25% w/v and cryoprotectant (Glucose) of 15% w/v are added to aqueous phase containing microparticles and allowed to dissolve (washing of particles using centrifugation or tangential flow filtration can be done as an optional step prior to adding gel-forming polymer). The emulsion is lyophilized for 24 hours to obtain the final product. Once the microparticle-gel is injected in brain, the drug released from the dispersed PLGA particles will complex with the outer layer of Poloxamer (gel-forming material) to in situ form <100 nm sized Poloxamer-drug nanocomplexes (FIG. 14B).

### Example 15: Development of Injectable Gel Microstructure Consisting of 3-5 μm PLGA Particles Loaded with Drug Piperlongumine (10 wt. %) and Surface Coated with Pluronic In Situ Releasing Pluronic-PL (Piperlongumine) Nanocomplexes of Size <100 nm

In this example, preparation of microparticles of size 3-5 μm loaded with 10% Piperlongumine (PL) is described (FIG. 4, 5). 100 mg PLGA (75:25, 20 kDa) and 100 mg PLA (37 kDa) are dissolved in 2 ml Acetone and allowed to stir at 500 rpm for 45 minutes to 1 hour. 2% w/v PEG (20 kDa) is dissolved in 10 ml MilliQ water at 700-800 rpm, for 1 hour. PL (dissolved in suitable solvent) is added to PLGA solution and allowed to blend for 2 hours, following which it is emulsified in PEG solution. Following emulsification, solution is allowed to stir for 2-4 hours to evaporate the solvent. The gel-forming polymer (Pluronic) of 25% w/v and cryoprotectant (Glucose) of 15% w/v are added to aqueous phase containing microparticles and allowed to dissolve (washing of particles using centrifugation or tangential flow filtration can be done as an optional step prior to adding gel-forming polymer). The emulsion is lyophilized for 24 hours to obtain the final product. Once the microparticle-gel is injected in brain, the drug released from the dispersed PLGA particles will complex with the outer layer of Pluronic (gel-forming material) to in situ form <100 nm sized Pluronic-drug nanocomplexes (FIG. 13D).

### Example 16: Development of Injectable Gel Microstructure Consisting of 3-5 μm PLGA Particles Loaded with Drug Piperlongumine (10 wt. %) and Surface Coated with Kolliphor In Situ Releasing Kolliphor-PL (Piperlongumine) Nanocomplexes of Size <100 nm

In this example, preparation of microparticles of size 3-5 μm loaded with 10% Piperlongumine (PL) is described (FIG. 4, 5). 100 mg PLGA (75:25, 20 kDa) and 100 mg PLA (37 kDa) are dissolved in 2 ml Acetone and allowed to stir at 500 rpm for 45 minutes to 1 hour. 2% w/v PEG (20 kDa) is dissolved in 10 ml MilliQ water at 700-800 rpm, for 1 hour. PL (dissolved in suitable solvent) is added to PLGA solution and allowed to blend for 2 hours, following which it is emulsified in PEG solution. Following emulsification, solution is allowed to stir for 2-4 hours to evaporate the solvent. The gel-forming polymer (Kolliphor) of 25% w/v and cryoprotectant (Glucose) of 15% w/v are added to aqueous phase containing microparticles and allowed to dissolve (washing of particles using centrifugation or tangential flow filtration can be done as an optional step prior to adding gel-forming polymer). The emulsion is lyophilized for 24 hours to obtain the final product. Once the microparticle-gel is injected in brain, the drug released from the dispersed PLGA particles will complex with the outer layer of Kolliphor (gel-forming material) to in situ form <100 nm sized Kolliphor-drug nanocomplexes (FIG. 13E).

### Example 17: Development of Injectable Gel Microstructure Consisting of 3-5 μm PLGA Particles Loaded with Drug Piperlongumine (10 wt. %) and Surface Coated with Soluplus In Situ Releasing Soluplus-PL (Piperlongumine) Nanocomplexes of Size <100 nm

In this example, preparation of microparticles of size 3-5 μm loaded with 10% Piperlongumine (PL) is described (FIG. 4, 5). 100 mg PLGA (75:25, 20 kDa) and 100 mg PLA (37 kDa) are dissolved in 2 ml Acetone and allowed to stir at 500 rpm for 45 minutes to 1 hour. 2% w/v PEG (20 kDa) is dissolved in 10 ml MilliQ water at 700-800 rpm, for 1 hour. PL (dissolved in suitable solvent) is added to PLGA solution and allowed to blend for 2 hours, following which it is emulsified in PEG solution. Following emulsification, solution is allowed to stir for 2-4 hours to evaporate the solvent. The gel-forming polymer (Soluplus) of 25% w/v and cryoprotectant (Glucose) of 15% w/v are added to aqueous phase containing microparticles and allowed to dissolve (washing of particles using centrifugation or tangential flow filtration can be done as an optional step prior to adding gel-forming polymer). The emulsion is lyophilized for 24 hours to obtain the final product. Once the microparticle-gel is injected in brain, the drug released from the dispersed PLGA particles will complex with the outer layer of Soluplus (gel-forming material) to in situ form <100 nm sized Soluplus-drug nanocomplexes (FIG. 13F).

### Example 18: Development of Injectable Gel Microstructure Consisting of 100-500 μm PLGA Particles Loaded with Drug BCNU (20 wt. %) and Surface Coated with PEG 400 In Situ Releasing PEG 400-BCNU Nanocomplexes of Size <100 nm

In this example, preparation of microparticles of size 100-500 μm loaded with 20 wt. % BCNU is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. BCNU (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (PEG 400) of 50% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of PEG 400 (gel-forming material) to in situ form <100 nm sized PEG-drug nanocomplexes. Characterization of these microparticles using SEM is shown in FIG. 10A-F.

### Example 19: Development of Injectable Gel Microstructure Consisting of 100-500 μm PLGA Particles Loaded with Drug Temozolomide (18 wt. %) and Surface Coated with PEG 400 In Situ Releasing PEG 400-Temozolomide (TMZ) Nanocomplexes of Size <100 nm

In this example, preparation of microparticles of size 100-500 μm loaded with 18 wt. % Temozolomide (TMZ) is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. TMZ (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (PEG 400) of 50% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of PEG 400 (gel-forming material) to in situ form <100 nm sized PEG-drug nanocomplexes (FIG. 15A). Characterization of these microparticles using SEM is shown in FIG. 11A-F.

### Example 20: Development of Injectable Gel Microstructure Consisting of 100-500 μm PLGA Particles Loaded with Drug Temozolomide (18 wt. %) and Surface Coated with PEG 6 kDa In Situ Releasing PEG 6 kDa-Temozolomide (TMZ) Nanocomplexes of Size <100 nm

In this example, preparation of microparticles of size 100-500 μm loaded with 18 wt. % Temozolomide (TMZ) is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. TMZ (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (PEG 6 kDa) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of PEG 6 kDa (gel-forming material) to in situ form <100 nm sized PEG-drug nanocomplexes (FIG. 15E).

### Example 21: Development of Injectable Gel Microstructure Consisting of 100-500 μm PLGA Particles Loaded with Drug Temozolomide (18 wt. %) and Surface Coated with PEG 20 kDa In Situ Releasing PEG 20 kDa-Temozolomide (TMZ) Nanocomplexes of Size <100 nm

In this example, preparation of microparticles of size 100-500 μm loaded with 18 wt. % Temozolomide (TMZ) is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. TMZ (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (PEG 20 kDa) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of PEG 20 kDa (gel-forming material) to in situ form <100 nm sized PEG-drug nanocomplexes (FIG. 16A).

### Example 22: Development of Injectable Gel Microstructure Consisting of 100-500 μm PLGA Particles Loaded with Drug Temozolomide (18 wt. %) and Surface Coated with Poloxamer In Situ Releasing Poloxamer-Temozolomide (TMZ) Nanocomplexes of Size <100 nm

In this example, preparation of microparticles of size 100-500 μm loaded with 18 wt. % Temozolomide (TMZ) is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. TMZ (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (Poloxamer) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of Poloxamer (gel-forming material) to in situ form <100 nm sized Poloxamer-drug nanocomplexes (FIG. 14C).

### Example 23: Development of Injectable Gel Microstructure Consisting of 100-500 μm PLGA Particles Loaded with Drug Temozolomide (18 wt. %) and Surface Coated with Pluronic In Situ Releasing Pluronic-Temozolomide (TMZ) Nanocomplexes of Size <100 nm

In this example, preparation of microparticles of size 100-500 μm loaded with 18 wt. % Temozolomide (TMZ) is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. TMZ (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (Pluronic) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of Pluronic (gel-forming material) to in situ form <100 nm sized Pluronic-drug nanocomplexes (FIG. 16E).

### Example 24: Development of Injectable Gel Microstructure Consisting of 100-500 μm PLGA Particles Loaded with Drug Temozolomide (18 wt. %) and Surface Coated with Kolliphor In Situ Releasing Kolliphor-Temozolomide (TMZ) Nanocomplexes of Size <100 nm

In this example, preparation of microparticles of size 100-500 μm loaded with 18 wt. % Temozolomide (TMZ) is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. TMZ (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (Kolliphor) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in the brain, the drug released from the dispersed PLGA particles will complex with the outer layer of Kolliphor (gel-forming material) to in situ form <100 nm sized Kolliphor-drug nanocomplexes (FIG. 17A).

### Example 25: Development of Injectable Gel Microstructure Consisting of 100-500 μm PLGA Particles Loaded with Drug Temozolomide (18 wt. %) and Surface Coated with Soluplus In Situ Releasing Soluplus-Temozolomide (TMZ) Nanocomplexes of Size <100 nm

In this example, preparation of microparticles of size 100-500 μm loaded with 18 wt. % Temozolomide (TMZ) is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. TMZ (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (Soluplus) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of Soluplus (gel-forming material) to in situ form <100 nm sized Soluplus-drug nanocomplexes (FIG. 17E).

### Example 26: Development of Injectable Gel Microstructure Consisting of 100-500 μm PLGA Particles Loaded with Drug 5-Flourouracil (18 wt. %) and Surface Coated with PEG 400 In Situ Releasing PEG 400-5-Flourouracil (5-FU) Nanocomplexes of Size <100 nm

In this example, preparation of microparticles of size 100-500 μm loaded with 18 wt. % 5-FU is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. 5-Flourouracil (5-FU) (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (PEG 400) of 50% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of PEG 400 (gel-forming material) to in situ form <100 nm sized PEG-drug nanocomplexes (FIG. 15B).

### Example 27: Development of Injectable Gel Microstructure Consisting of 100-500 μm PLGA Particles Loaded with Drug 5-Flourouracil (18 wt. %) and Surface Coated with PEG 6 kDa In Situ Releasing PEG 6 kDa-5-Flourouracil (5-FU) Nanocomplexes of Size <100 nm

In this example, preparation of microparticles of size 100-500 μm loaded with 18 wt. % 5-FU is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. 5-Flourouracil (5-FU) (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (PEG 6 kDa) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of PEG 6 kDa (gel-forming material) to in situ form <100 nm sized PEG-drug nanocomplexes (FIG. 15F).

### Example 28: Development of Injectable Gel Microstructure Consisting of 100-500 μm PLGA Particles Loaded with Drug 5-Flourouracil (18 wt. %) and Surface Coated with PEG 20 kDa In Situ Releasing PEG 20 kDa-5-Flourouracil (5-FU) Nanocomplexes of Size <100 nm

In this example, preparation of microparticles of size 100-500 μm loaded with 18 wt. % 5-FU is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. 5-Flourouracil (5-FU) (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (PEG 20 kDa) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of PEG 20 kDa (gel-forming material) to in situ form <100 nm sized PEG-drug nanocomplexes (FIG. 16B).

### Example 29: Development of Injectable Gel Microstructure Consisting of 100-500 μm PLGA Particles Loaded with Drug 5-Flourouracil (18 wt. %) and Surface Coated with Pluronic In Situ Releasing Pluronic-5-Flourouracil (5-FU) Nanocomplexes of Size <100 nm

In this example, preparation of microparticles of size 100-500 μm loaded with 18 wt. % 5-FU is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. 5-Flourouracil (5-FU) (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (Pluronic) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of Pluronic (gel-forming material) to in situ form <100 nm sized Pluronic-drug nanocomplexes (FIG. 16F).

### Example 30: Development of Injectable Gel Microstructure Consisting of 100-500 μm PLGA Particles Loaded with Drug 5-Flourouracil (18 wt. %) and Surface Coated with Kolliphor In Situ Releasing Kolliphor-5-Flourouracil (5-FU) Nanocomplexes of Size <100 nm

In this example, preparation of microparticles of size 100-500 μm loaded with 18 wt. % 5-FU is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. 5-Flourouracil (5-FU) (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (Kolliphor) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in the brain, the drug released from the dispersed PLGA particles will complex with the outer layer of Kolliphor (gel-forming material) to in situ form <100 nm sized Kolliphor-drug nanocomplexes (FIG. 17B).

### Example 31: Development of Injectable Gel Microstructure Consisting of 100-500 μm PLGA Particles Loaded with Drug 5-Flourouracil (18 wt. %) and Surface Coated with Soluplus In Situ Releasing Soluplus-5-Flourouracil (5-FU) Nanocomplexes of Size <100 nm

In this example, preparation of microparticles of size 100-500 μm loaded with 18 wt. % 5-FU is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. 5-Flourouracil (5-FU) (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (Soluplus) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of Soluplus (gel-forming material) to in situ form <100 nm sized Soluplus-drug nanocomplexes (FIG. 17F).

### Example 32: Development of Injectable Gel Microstructure Consisting of 100-500 μm PLGA Particles Loaded with Drug Doxorubicin (18 wt. %) and Surface Coated with PEG 400 In Situ Releasing PEG 400-Doxorubicin (Dox) Nanocomplexes of Size <100 nm

In this example, preparation of microparticles of size 100-500 μm loaded with 18 wt. % Doxorubicin (Dox) is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. Doxorubicin (Dox) (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (PEG 400) of 50% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of PEG 400 (gel-forming material) to in situ form <100 nm sized PEG-drug nanocomplexes (FIG. 15C).

### Example 33: Development of Injectable Gel Microstructure Consisting of 100-500 μm PLGA Particles Loaded with Drug Doxorubicin (18 wt. %) and Surface Coated with PEG 6 kDa In Situ Releasing PEG 6 kDa-Doxorubicin (Dox) Nanocomplexes of Size <100 nm

In this example, preparation of microparticles of size 100-500 μm loaded with 18 wt. % Doxorubicin (Dox) is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. Doxorubicin (Dox) (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (PEG 6 kDa) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of PEG 6 kDa (gel-forming material) to in situ form <100 nm sized PEG-drug nanocomplexes (FIG. 15G).

### Example 34: Development of Injectable Gel Microstructure Consisting of 100-500 μm PLGA Particles Loaded with Drug Doxorubicin (18 wt. %) and Surface Coated with PEG 20 kDa In Situ Releasing PEG 20 kDa-Doxorubicin (Dox) Nanocomplexes of Size <100 nm

In this example, preparation of microparticles of size 100-500 μm loaded with 18 wt. % Doxorubicin (Dox) is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. Doxorubicin (Dox) (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (PEG 20 kDa) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of PEG 20 kDa (gel-forming material) to in situ form <100 nm sized PEG-drug nanocomplexes (FIG. 16C).

### Example 35: Development of Injectable Gel Microstructure Consisting of 100-500 μm PLGA Particles Loaded with Drug Doxorubicin (18 wt. %) and Surface Coated with Pluronic In Situ Releasing Pluronic-Doxorubicin (Dox) Nanocomplexes of Size <100 nm

In this example, preparation of microparticles of size 100-500 μm loaded with 18 wt. % Doxorubicin (Dox) is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. Doxorubicin (Dox) (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (Pluronic) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of Pluronic (gel-forming material) to in situ form <100 nm sized Pluronic-drug nanocomplexes (FIG. 16G).

### Example 36: Development of Injectable Gel Microstructure Consisting of 100-500 μm PLGA Particles Loaded with Drug Doxorubicin (18 wt. %) and Surface Coated with Kolliphor In Situ Releasing Kolliphor-Doxorubicin (Dox) Nanocomplexes of Size <100 nm

In this example, preparation of microparticles of size 100-500 μm loaded with 18 wt. % Doxorubicin (Dox) is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. Doxorubicin (Dox) (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (Kolliphor) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of Kolliphor (gel-forming material) to in situ form <100 nm sized Kolliphor-drug nanocomplexes (FIG. 17C).

### Example 37: Development of Injectable Gel Microstructure Consisting of 100-500 μm PLGA Particles Loaded with Drug Doxorubicin (18 wt. %) and Surface Coated with Soluplus In Situ Releasing Soluplus-Doxorubicin (Dox) Nanocomplexes of Size <100 nm

In this example, preparation of microparticles of size 100-500 μm loaded with 18 wt. % Doxorubicin (Dox) is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. Doxorubicin (Dox) (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (Soluplus) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of Soluplus (gel-forming material) to in situ form <100 nm sized Soluplus-drug nanocomplexes (FIG. 17G).

### Example 38: Development of Injectable Gel Microstructure Consisting of 100-500 μm PLGA Particles Loaded with Drug Gemcitabine (18 wt. %) and Surface Coated with PEG 400 In Situ Releasing PEG 400-Gemcitabine (Gem) Nanocomplexes of Size <100 nm

In this example, preparation of microparticles of size 100-500 μm loaded with 18 wt. % Gemcitabine (Gem) is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. Gemcitabine (Gem) (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (PEG 400) of 50% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of PEG 400 (gel-forming material) to in situ form <100 nm sized PEG-drug nanocomplexes (FIG. 15D).

### Example 39: Development of Injectable Gel Microstructure Consisting of 100-500 μm PLGA Particles Loaded with Drug Gemcitabine (18 wt. %) and Surface Coated with PEG 6 kDa In Situ Releasing PEG 6 kDa-Gemcitabine (Gem) Nanocomplexes of Size <100 nm

In this example, preparation of microparticles of size 100-500 μm loaded with 18 wt. % Gemcitabine (Gem) is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. Gemcitabine (Gem) (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (PEG 6 kDa) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of PEG 6 kDa (gel-forming material) to in situ form <100 nm sized PEG-drug nanocomplexes (FIG. 15H).

### Example 40: Development of Injectable Gel Microstructure Consisting of 100-500 μm PLGA Particles Loaded with Drug Gemcitabine (18 wt. %) and Surface Coated with PEG 20 kDa In Situ Releasing PEG 20 kDa-Gemcitabine (Gem) Nanocomplexes of Size <100 nm

In this example, preparation of microparticles of size 100-500 μm loaded with 18 wt. % Gemcitabine (Gem) is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. Gemcitabine (Gem) (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (PEG 20 kDa) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of PEG 20 kDa (gel-forming material) to in situ form <100 nm sized PEG-drug nanocomplexes (FIG. 16D).

### Example 41: Development of Injectable Gel Microstructure Consisting of 100-500 μm PLGA Particles Loaded with Drug Gemcitabine (18 wt. %) and Surface Coated with Pluronic In Situ Releasing Pluronic-Gemcitabine (Gem) Nanocomplexes of Size <100 nm

In this example, preparation of microparticles of size 100-500 μm loaded with 18 wt. % Gemcitabine (Gem) is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. Gemcitabine (Gem) (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (Pluronic) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of Pluronic (gel-forming material) to in situ form <100 nm sized Pluronic-drug nanocomplexes (FIG. 16H).

### Example 42: Development of Injectable Gel Microstructure Consisting of 100-500 μm PLGA Particles Loaded with Drug Gemcitabine (18 wt. %) and Surface Coated with Kolliphor In Situ Releasing Kolliphor-Gemcitabine (Gem) Nanocomplexes of Size <100 nm

In this example, preparation of microparticles of size 100-500 μm loaded with 18 wt. % Gemcitabine (Gem) is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. Gemcitabine (Gem) (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (Kolliphor) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in the brain, the drug released from the dispersed PLGA particles will complex with the outer layer of Kolliphor (gel-forming material) to in situ form <100 nm sized Kolliphor-drug nanocomplexes (FIG. 17D).

### Example 43: Development of Injectable Gel Microstructure Consisting of 100-500 μm PLGA Particles Loaded with Drug Gemcitabine (18 wt. %) and Surface Coated with Soluplus In Situ Releasing Soluplus-Gemcitabine (Gem) Nanocomplexes of Size <100 nm

In this example, preparation of microparticles of size 100-500 μm loaded with 18 wt. % Gemcitabine (Gem) is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. Gemcitabine (Gem) (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (Soluplus) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of Soluplus (gel-forming material) to in situ form <100 nm sized Soluplus-drug nanocomplexes (FIG. 17H).

### Example 44: Development of Injectable Gel Microstructure Consisting of 100-500 μm PLGA Particles Loaded with Drug Paclitaxel (18 wt. %) and Surface Coated with PEG 400 In Situ Releasing PEG 400-Paclitaxel (PTX) Nanocomplexes of Size <100 nm

In this example, preparation of microparticles of size 100-500 μm loaded with 18 wt. % Gemcitabine (Gem) is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. Gemcitabine (Gem) (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (PEG 400) of 50% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of PEG 400 (gel-forming material) to in situ form <100 nm sized PEG-drug nanocomplexes (FIG. 18A).

### Example 45: Development of Injectable Gel Microstructure Consisting of 100-500 μm PLGA Particles Loaded with Drug Paclitaxel (18 wt. %) and Surface Coated with PEG 6 kDa In Situ Releasing PEG 6 kDa-Paclitaxel (PTX) Nanocomplexes of Size <100 nm

In this example, preparation of microparticles of size 100-500 μm loaded with 18 wt. % Gemcitabine (Gem) is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. Gemcitabine (Gem) (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (PEG 6 kDa) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of PEG 6 kDa (gel-forming material) to in situ form <100 nm sized PEG-drug nanocomplexes (FIG. 18B).

### Example 46: Development of Injectable Gel Microstructure Consisting of 100-500 μm PLGA Particles Loaded with Drug Paclitaxel (18 wt. %) and Surface Coated with PEG 20 kDa In Situ Releasing PEG 20 kDa-Paclitaxel (PTX) Nanocomplexes of Size <100 nm

In this example, preparation of microparticles of size 100-500 μm loaded with 18 wt. % Gemcitabine (Gem) is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. Gemcitabine (Gem) (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (PEG 20 kDa) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of PEG 20 kDa (gel-forming material) to in situ form <100 nm sized PEG-drug nanocomplexes (FIG. 18C).

### Example 47: Development of Injectable Gel Microstructure Consisting of 100-500 μm PLGA Particles Loaded with Drug Paclitaxel (18 wt. %) and Surface Coated with Pluronic In Situ Releasing Pluronic-Paclitaxel (PTX) Nanocomplexes of Size <100 nm

In this example, preparation of microparticles of size 100-500 μm loaded with 18 wt. % Gemcitabine (Gem) is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. Gemcitabine (Gem) (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (Pluronic) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of Pluronic (gel-forming material) to in situ form <100 nm sized Pluronic-drug nanocomplexes (FIG. 18D).

### Example 48: Development of Injectable Gel Microstructure Consisting of 100-500 μm PLGA Particles Loaded with Drug Paclitaxel (18 wt. %) and Surface Coated with Kolliphor In Situ Releasing Kolliphor-Paclitaxel (PTX) Nanocomplexes of Size <100 nm

In this example, preparation of microparticles of size 100-500 μm loaded with 18 wt. % Gemcitabine (Gem) is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. Gemcitabine (Gem) (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (Kolliphor) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of Kolliphor (gel-forming material) to in situ form <100 nm sized Kolliphor-drug nanocomplexes (FIG. 18E).

### Example 49: Development of Injectable Gel Microstructure Consisting of 100-500 μm PLGA Particles Loaded with Drug Paclitaxel (18 wt. %) and Surface Coated with Soluplus In Situ Releasing Soluplus-Paclitaxel (PTX) Nanocomplexes of Size <100 nm

In this example, preparation of microparticles of size 100-500 μm loaded with 18 wt. % Gemcitabine (Gem) is described (FIG. 6, 7). 100 mg PLGA (75:25) is dissolved in 2 ml Dichloromethane (DCM) and allowed to stir at 500 rpm for 30 minutes to 1 hour. 2.5% w/v PVA is dissolved in 100 ml MilliQ water at 90° C., 700-800 rpm, for 1 hour. Gemcitabine (Gem) (dissolved in suitable solvent) is added to PLGA solution and allowed to blend for 5 minutes at 500 rpm, following which it is added to the PVA solution using layering method (polymer-drug solution containing syringe added directly into the PVA solution) at 515 rpm speed using overhead stirring. The drug polymer-solution is emulsified in the aqueous phase using layering, wherein the needle containing polymer-drug solution is injected slowly into the aqueous phase (after placing the needle at the bottom of the beaker containing the aqueous phase). Following emulsification, solution is allowed to stir for 2-4 hours to evaporate the solvent. The gel-forming polymer (Soluplus) of 25% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 48 hours to obtain the final product. Once the microparticle-gel is injected in brain, the drug released from the dispersed PLGA particles will complex with the outer layer of Soluplus (gel-forming material) to in situ form <100 nm sized Soluplus-drug nanocomplexes (FIG. 18F).

### Example 50: Development of Injectable Gel Microstructure Consisting of 100-500 μm PLGA Particles Loaded with Temozolomide (TMZ) (18 wt. % Drug Loading) Drug Using High Pressure Homogenization (HPH) Technique

In this example, preparation of microparticles of size 100-500 μm loaded with drug is described. 100 mg PLGA (75:25) is dissolved in 2 ml Dichloromethane (DCM) and allowed to stir at 500 rpm for 30 minutes to 1 hour. The drug Temozolomide (TMZ) of 18 wt % drug loading, is dissolved in polymer solution for 2 hours under stirring at 500 rpm. 2.5 wt. % PVA is dissolved in 100 ml MilliQ water at 90° C., 700-800 rpm, for 1 hour. The PVA aqueous solution is added to the reservoir of the HPH system and under pressure conditions of 15000 psi for 3 cycles, the polymer-drug solution is added to form microparticles containing solution. The gel-forming polymer (PEG 400) of 50% w/v and cryoprotectant (Mannitol) of 15% w/v are added to aqueous phase containing microparticles and allowed to dissolve (washing the particles initially with 2% PVA solution, followed by 1% PVA solution and finally with deionized water, using centrifugation or tangential flow filtration can be performed as an optional step prior to adding the gel-forming polymer). The emulsion is lyophilized for 24 hours to obtain the final product. Once the microparticle-gel is injected in brain, the drug released from the dispersed PLGA particles will complex with the outer layer of PEG 400 (gel-forming material) to in situ form <100 nm sized PEG-drug nanocomplexes.

### Example 51: Size Analysis of PEG 400-BCNU Nanocomplexes (BCNU-NC-33) Formed after BCNU Release from Microparticles and its Interaction with Outer PEG 400 Gel Coating

The size analysis of BCNU-NC-33 nancomplexes were analyzed in vitro, by dispersion of BCNU-3 μm particles in a gel pool of PEG 400 and allowed to blend for 4 hours. After 4 hours, the contents are centrifuged at 2000 rpm for 10 minutes and the supernatant is analyzed for its size. The morphology and size of drug-polymer nanocomplexes formed in situ (FIG. 19A-D) from microparticles (FIG. 19E-H) and its size being between ˜50-80 nm is depicted using Transmission Electron Microscopy (TEM). FIG. 191 shows the Raman spectroscopy confirming the presence of drug BCNU in BCNU-NC-33 nanocomplexes formed after the complexation of BCNU with the outer gel layer of PEG 400. Further confirmation of the presence of BCNU in BCNU-NC-33 nanocomplexes using High Pressure Liquid Chromatography (HPLC) as depicted in FIG. 19J.

### Example 52: Ex Vivo Diffusion of ICG (Indocyanine) Loaded Nanocomplexes (ICG-NC-33) and ICG-0.25 μm Particles without Gel Coating in Goat Brain Phantom Using Near Infrared (NIR) Imaging

ICG loaded microstructures (in the form of injectable gels) are injected in left and right hemispheres of a goat brain phantom and imaged using NIR over 4 hours (FIG. 20 A-G). ICG loaded nanocomplexes (ICG-NC-33) showed >4 cm longitudinal diffusion and 3.4 cm lateral diffusion by 5^{th} hour, whereas ICG-0.25 μm particles without gel coating showed 1.47 cm longitudinal and 0.83 cm lateral diffusion by 5^{th} hour. ICG-NC-33 showed >3.5 cm increased in diffusion compared to ICG-0.25 μm particles without gel coating, showing the influence of size of microparticles for diffusion.

### Example 53: Ex Vivo Diffusion of Iodine Loaded Nanocomplexes (Iodine-NC-33), Iodine-0.25 μm Particles without Gel Coating, Iodine-0.25 μm Particles Coated with Gel, Iodine-3 μm Particles without Gel Coating and Iodine-3 μm Particles Coated with Gel in Goat Brain Phantom Using CT Imaging

Iodine loaded microstructures (in the form of injectable gels) are injected in left and right hemispheres of a goat brain phantom and imaged over 4 hours using CT (FIG. 21 (A-E)). Free iodine diffused 1.6 cm by 3^{rd} hour, Iodine loaded nanocomplexes (Iodine-NC-33) showed >3.5 cm diffusion by 4^{th} hour, Iodine-0.25 μm particles without gel coating showed 1.5 cm, Iodine-0.25 μm particles coated with gel showed 2.62 cm, Iodine-3 μm particles without gel coating showed 0.43 cm and Iodine-3 μm particles coated with gel showed 0.86 cm by the 4^{th} hour. Iodine-NC-33 nanocomplexes also showed >3 cm increased in diffusion compared to larger sized microstructures.

### Example 54: In Vitro Cytotoxicity of BCNU and PL Loaded Microstructures in C6 and T98G Cells

Cells of seeding density 4×10⁴ cells/well were treated with concentrations from 1 μM-100 μM of BCNU or PL loaded microstructures 24 hours post seeding. MTT assay was performed 96 hours post treatment. FIG. 22 (A-D) shows the cytotoxicity of free BCNU, BCNU-NC-33, BCNU-0.25 μm particles coated with gel, BCNU-3 μm particles coated with gel and free PL, PL-NC-33, PL-0.25 μm particles coated with gel and PL-3 μm particles coated with gel C6 (rat glioma) and T98G (human glioma) cells. Most cytotoxicity was observed by polymer—BCNU nanocomplexes (BCNU-NC-33) with ˜15% cells viable at 100 μM and BCNU-3 μm particles coated with gel was the least cytotoxic on C6 cells with ˜25% viability observed at 100 μM concentration (FIG. 22A). BCNU-0.25 μm particles coated with gel showed ˜23% and free BCNU showed ˜20% cell viability at the highest concentration. Whereas, PL based systems showed a higher level of cytotoxicity with ˜3%, 2%, ˜9% and ˜7% cells viable at 100 μM concentration of free PL, PL-NC-33, PL-0.25 μm particles coated with gel and PL-3 μm particles coated with gel respectively on C6 cells (FIG. 22C). With radiation, the cell viability of non-drug treated controls reduced by ˜60%. Whereas, in T98G cells, BCNU loaded microstructures did not show a significant effect (FIG. 22B) with minimum cell viability at 80% at highest concentration. Whereas, PL based systems showed a significant difference in cytotoxicity levels, with only 16.6%, ˜5%, 22.2% and 27.6% cells remaining at 100 μM for free PL, PL-NC-33, PL-0.25 μm particles coated with gel and PL-3 μm particles coated with gel (FIG. 22D). This shows the improved therapeutic effect of PL in resistant cell lines.

### Example 55: In Vivo Drug Concentration Analysis of Free BCNU, BCNU-NC-33, BCNU-0.25 μm Particles Coated with Gel and BCNU-3 μm Particles Coated with Gel in Healthy Rat Brain

200-250 g Wistar rats (female) were selected for the study. Respective microparticle gel was injected (10 μl/1 mg BCNU) injected at coordinates 2 mm lateral, 2 mm anterior and 2 mm depth using stereotactic equipment. Animals were euthanized 3, 7 and 15 days post injection, brains were excised and stored at −80° C. Each brain was sliced into 1 mm sections and each section was homogenized in 1 ml MilliQ water (pH 4) and precipitated using 10 ml Ethanol. The samples are centrifuged at 10000 rpm for 15 minutes for drug extraction (repeated 4 times). The solvents are evaporated using a vacuum centrifuge and then remnant pellets are analyzed for their drug content using HPLC. Free BCNU showed the least amount of drug of 15% on 3^{rd} day in brain which degraded to 3.9% on day-15 (FIG. 23). BCNU-NC-33 nanocomplexes showed 82.8% drug on day-3 which reduced to 24.6% on day-15. Similarly, BCNU-0.25 μm particles with gel coating and BCNU-3 μm particles with gel coating showed 90.4%, 86.1% drug on day-3 and 55.6%, 73.6% drug on day-15 respectively. BCNU-3 μm particles with gel coating showed maximum drug concentration in brain with ˜70% and ˜50% higher drug content compared free BCNU and BCNU-NC-33 respectively.

### Example 56: In Vivo Anti-Tumor Efficacy Study of BCNU-NC-33, BCNU-0.25 μm Particles Coated with Gel and BCNU-3 μm Particles Coated with Gel in Orthotopic Rat Tumor Model

Female Wistar rats of 150-200 g were selected for the study. Briefly, each animal was anesthetized with Ketamine and Xylazine combination. Post shaving, an incision of 1.5 cm was made on the skull and a drill hole of 0.5 mm was made. 1×10⁶ cells (10p) was injected at coordinated 2 mm lateral, 2 mm anterior and 2 mm depth. 7 days post tumor induction, a repeat surgery was performed and BCNU-33 or BCNU-250 gel was injected at respective coordinates. MRI was performed at periodic intervals and tumor volume was calculated. MR imaging showed that untreated group tumor volume increased from 15.07±9.7 mm³ to 108.9±14.1 mm³ from day-8 to day-16 post tumor inoculation (FIG. 24A). Whereas, BCNU-0.25 μm particles coated with gel injected animals showed a decrease in tumor volume from 29.27±2.5 mm³ day-1 post injection, to 27.08±2 mm³ on day-3 and 4.74 mm³ was remaining on day-7 post NP-gel injection (FIG. 24C). BCNU-0.25 μm particles coated with gel showed a decrease in tumor volume by 49.94%, 16.38% by day-1, to 98.99%, 95.46% by day-15 compared to untreated control and free BCNU (FIG. 24B) respectively. BCNU-3 μm particles coated with gel showed a decrease in tumor volume by 72.37%, 3.88% by day-1, to 96.92%, 86.05% by day-15 post injection (FIG. 24D). Faster reduction in tumor volume by BCNU-0.25 μm particles coated with gel was observed compared to BCNU-3 μm particles coated with gel, due to immediate release by smaller sized particles (FIG. 24E).

The foregoing description of the various embodiments is provided to enable any person skilled in art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein, and instead the claims should be accorded the widest scope consistent with the principles and novel features disclosed herein.

While the invention has been described with reference to a preferred embodiment, it is apparent that variations and modifications will occur without departing the spirit and scope of the invention. It is therefore contemplated that the present disclosure covers any and all modifications, variations or equivalents that fall within the scope of the basic underlying principles disclosed above.

1. An injectable microparticle system comprising drug-loaded polymeric microparticles, wherein the drug-loaded polymeric microparticles are coated with an outer polymer gel layer enabling in-situ formation and releasing drug-polymer nanocomplexes of size <100 nm.

2. The system as claimed in claim 1, wherein the drug-loaded polymeric microparticle is made of biodegradable and biocompatible polymer.

3. The system as claimed in claim 2, wherein the biodegradable and biocompatible polymer is selected from Poly lactic-co-glycolic acid (PLGA), Poly lactic acid (PLA), Polyvinyl alcohol (PVA), Poly caprolactam (PCL), or combination thereof.

4. The system as claimed in claim 1, wherein the size of drug-loaded polymeric microparticle is in a range of 0.25-1000 microns.

5. The system as claimed in claim 1, wherein a gel-forming polymer of outer polymer gel layer is selected from Polyethylene glycol having molecular weight ranging from 300-40000 Da, Poloxamer, Polyoxyl 15 hydroxystearate, Polyoxyl 35 Castor oil, Polysiloxane, Polysorbate 20, Polysorbate 80, Pluronic® (block copolymer of polyethylene oxide and polypropylene oxide), Soluplus® (graft copolymer of polyethylene glycol, polyvinylcaprolactam and polyvinylacetate), Kolliphor® (mixture of castor oil and ethylene oxide), or Polyvinyl alcohol.

6. The system as claimed in claim 1, wherein the ratio of polymer of the drug-loaded polymeric microparticles to gel-forming polymer is in a range of 1:0.1 to 1:50 w/w %.

7. The system as claimed in claim 1, wherein a drug in the drug-loaded microparticles is selected from temozolomide, carmustine (BCNU), lomustine (CCNU), piperlongumine (PL), paclitaxel, cetuximab, irinotecan, everolimus, carboplatin, platinums, etoposide, methotrexate, Ara-c, pemetrexed, thiotepa, docetaxel, 5-flurouracil (5FU), 6-thioguanine (6TG), cisplatin, topotecan, bevacizumab, gemcitabine, doxorubicin, D-actinomycin, epirubicin, procarbazine, vincristine, tyrosine kinase inhibitors, kinase inhibitors, photodynamic therapy drugs, mTHPC, porphyrin, staurosporine, midostaurin, therapeutic proteins, GMCSF (Granulocyte-macrophage colony-stimulating factor), BDNF (Brain-Derived Neutrotrophic Factor), GCSF (Granulocyte colony-stimulating factor), MCSF (macrophage colony-stimulating factor), PEGylated G-CSF, PEGylated GM-CSF and pharmaceutically acceptable salt, acid, or derivative thereof.

8. The system as claimed in claim 1, wherein the drug loaded microparticles are in the form of lyophilized or freeze-dried powder.

9. The system as claimed in claim 1, wherein the drug-polymer nanocomplexes penetrate the brain for >2 cm.

10. The system as claimed in claim 1, wherein the drug-polymer nanocomplexes is released in a sustained manner for a period of 15-30 days.

11. A method of preparing the injectable microparticle system as claimed in claim 1, comprising steps of:
(a) preparing a polymeric solution or a blend of polymeric solution by dissolving a polymer in an organic solvent;
(b) dissolving 1-50% wt/wt of a drug in the polymeric solution of step (a) to form a polymer-drug solution;
(c) forming a surfactant-aqueous solution by dissolving 0.1-50% w/v of a surfactant in water and stirring for 30 to 60 minutes;
(d) adding dropwise or directly injecting the polymer-drug solution of step (b) into the surfactant-aqueous solution of step (c) by stirring to form a micro-emulsion;
(e) evaporating the organic solvent from the micro-emulsion to obtain an aqueous phase having drug-loaded polymeric microparticles;
(f) optionally washing the aqueous phase of step (e) with 1% to 5% of Polyvinyl alcohol (PVA), and then with deionized water by centrifugation or tangential flow filtration;
(g) coating the drug-loaded microparticle of step (e) or (f) by adding 0.1-50% w/v of a gel-forming polymer and 1 to 50% w/v of cryoprotectants in the aqueous phase and homogenizing;
(h) lyophilizing the homogenized phase of step (g) to form drug-loaded polymeric microparticles powder coated and dispersed in gel forming polymer; and
(i) packing and sealing the lyophilized powder of step (h) in sterile condition.

12. The process as claimed in claim 11, wherein the blend of step (a) is prepared from the same polymer or two or three different polymers.

13. The process as claimed in claim 11, wherein the process optionally comprises a high pressure homogenization step to form the micro-emulsion at step (d).

14. The method as claimed in claim 11, wherein the polymer is selected Poly lactic-co-glycolic acid (PLGA), Poly lactic acid (PLA), Polyvinyl alcohol (PVA), Poly caprolactam (PCL), or combination thereof.

15. The method as claimed in claim 11, wherein the organic solvent is selected from dichloromethane (DCM), acetone, 1,4-Dioxane, chloroform, acetonitrile, dimethylformamide, ethyl acetate, methanol, ethanol, water, tetrahydrofuran, carbon tetrachloride, benzene, toluene, cyclohexanone, 2-nitropropane, or combination thereof.

16. The method as claimed in claim 11, wherein the drug is selected from temozolomide, carmustine (BCNU), lomustine (CCNU), piperlongumine (PL), paclitaxel, cetuximab, irinotecan, everolimus, carboplatin, platinums, etoposide, methotrexate, Ara-c, pemetrexed, thiotepa, docetaxel, 5-flurouracil (5FU), 6-thioguanine (6TG) cisplatin, topotecan, bevacizumab, gemcitabine, doxorubicin, D-actinomycin, epirubicin, procarbazine, vincristine, tyrosine kinase inhibitors, kinase inhibitors, photodynamic therapy drugs, mTHPC, porphyrin, staurosporine, midostaurin, therapeutic proteins, GMCSF (Granulocyte-macrophage colony-stimulating factor), BDNF (Brain-Derived Neutrotrophic Factor), GCSF (Granulocyte colony-stimulating factor), MCSF (macrophage colony-stimulating factor), PEGylated G-CSF, PEGylated GM-CSF and pharmaceutically acceptable salt, acid, or derivative thereof.

17. The method as claimed in claim 11, wherein the gel forming polymer is selected from Polyethylene glycol having molecular weight ranging from 300-40000 Da, Poloxamer, Polyoxyl 15 hydroxystearate, Polyoxyl 35 Castor oil, Polysiloxane, Polysorbate 20, Polysorbate 80, Pluronic® (block copolymer of polyethylene oxide and polypropylene oxide), Soluplus® (graft copolymer of polyethylene glycol, polyvinylcaprolactam and polyvinylacetate), Kolliphor® (mixture of castor oil and ethylene oxide), or Polyvinyl alcohol.

18. The method as claimed in claim 11, wherein the cryoprotectant is selected from Polyethylene glycol (PEG) having molecular weight ranging from 300-40000 Da, Propylene glycol, Polyvinylpyrrolidone (PVP), Polyvinyl alcohol (PVA), Glycerol, 2-methyl-2, 4-pentanediol (MPD), Sucrose, Glucose, Fructose, Trehalose, Mannitol, Proline, Sorbitol, Dextran, Poloxamer or Pluronic® (block copolymer of polyethylene oxide and polypropylene oxide).

19-21. (canceled)
